(19) United States
(12) Patent Application Publication
(10) Pub. No.: US 2003/0129597 A1
(43) Pub. Date: Jul. 10, 2003

(54) Identification of a novel endothelial-derived gene EG-1

(75) Inventor: Mai H. Nguyen, Los Angeles, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/029,137

(22) Filed: Dec. 19, 2001

(51) Int. Cl. C12Q 1/68; A01K 67/027; C07H 21/04; G01N 33/53; G01N 33/537; G01N 33/543; C07K 14/47

(52) U.S. Cl. 435/6; 435/69.1; 435/320.1; 435/325; 530/350; 536/23.5; 800/18; 435/7.92

(57) ABSTRACT

This invention provides a novel gene whose expression is upregulated during angiogenesis and/or tumorigenesis. Designated herein as EG-1, the gene provides a good target for modulators of angiogenesis and/or tumorigenesis. In addition, methods of inhibiting EG-1 and thereby inhibiting angiogenesis and metastasis are provided.

### STATEMENT AS TO RIGHTS TO INVENTIONS MADE UNDER FEDERALLY SPONSORED RESEARCH AND DEVELOPMENT

This invention was made with Government support under Grant No: CA69433, awarded by the National Institutes of Health. The Government of the United States of America may have certain rights in this invention.

### CROSS-REFERENCE TO RELATED APPLICATIONS

[Not Applicable]

### FIELD OF THE INVENTION

This invention pertains to the field of functional genomics. In particular, this invention pertains to the discovery of a novel endothelial-derived gene (designated EG-1) that is believed to be implicated in tumor angiogenesis.

### BACKGROUND OF THE INVENTION

The growth and metastasis of solid tumors is dependent on their ability to initiate and sustain new capillary growth, i.e. angiogenesis (Folkman (1995) N. Engl. J. Med. 333: 1757-1763). Angiogenesis is a complex multistep process which includes endothelial cell proliferation, migration and differentiation into tube-like structures. These steps involve changes in the expression of multiple growth factors, proteases and adhesion molecules in endothelial cells, as well as in supporting cells. Researchers have shown that endothelial cells lining established blood vessels have a very slow turnover time, whereas those lining tumor capillaries undergo rapid proliferation and differentiation. Although much has been discovered about adult angiogenesis, it was previously unclear whether abnormal angiogenesis such as that occurring in solid tumor growth involves different mechanisms from desirable angiogenesis which occurs in endometrial proliferation or in wound healing (Folkman (1995) Nature Med. 1: 27-31).

### SUMMARY OF THE INVENTION

In the past, efforts to identify the differences between the proliferating tumor endothelium and the normal quiescent endothelium have included antibody targeting (Huang et al. (1997) Science 275: 547-550), immunohistochemical analysis of known endothelial adhesion molecules (Nguyen et al. (1997) Am. J. Path. 150: 1307-1310), and phage display peptide libraries (Koivunen et al. (1999) Nat. Biotechnol. 17: 768-774). Differential RNA expression cloning has also been pursued in endothelial cells treated with TPA (Lee et al. (1998). Science 279: 1552-1555) and in endothelial cells derived from colorectal cancer (St. Crox et. al. (2000) Science 289: 1197-1202). In order to closely mimic a tumor environment, we have attempted to identify endothelial gene products expressed in response to a mixture of tumor derived growth factors found in tumor conditioned media. Toward this goal, we used a subtraction hybridization method called SSH (suppression subtractive hybridization, Diatchenko et al. (1996) Proc. Natl. Acad. Sci. USA. 93: 6025-6030). In HUVEC (human umbilical vein endothelial cell) populations exposed to tumor conditioned media for four hours, we have isolated approximately 300 up-regulated and another 300 down-regulated clones (Wang et al. (2000) Microvasc. Res. 59: 394-397). We named one of these differentially expressed genes EG-1 (endothelial-derived gene-1). In the present report, we show that EG-1 expression is seen in endothelial cells in several tissues and that its expression can be upregulated by growth stimulation induced by tumor conditioned medium as well as specific angiogenic factors. These results indicate that EG-1 plays a role in tumor angiogenesis. Consequently EG-1 makes a good target to screen for modulators of tissue angiogenesis and/or tumorigenesis. In addition, it is believed that EG-1 is a good therapeutic target.

Thus, in one embodiment, this invention provides an isolated nucleic acid comprising one or more of the following nucleic acids: (i) a nucleic acid that specifically hybridizes to a human EG-1 cDNA (coding region of SEQ ID NO:1) or a fragment thereof under stringent conditions and that is of sufficient length that said nucleic acid can uniquely indicate the presence or absence of a human EG-1 total genomic DNA pool, a total cDNA pool or a total mRNA pool sample from an endothelial cell; ii) a nucleic acid that encodes a human EG-1 polypeptide (SEQ ID NO:2); (iii) a nucleic acid that has the same sequence as a nucleic acid amplified from an endothelial cell mRNA template using PCR primers; (iv) a DNA encoding an mRNA that, when reverse transcribed, produces a human EG-1 cDNA (coding region of SEQ ID NO:1); v) a nucleic having 90 percent or greater sequence identity with a human EG-1 nucleic acid (coding region of SEQ ID NO:1) and encoding a polypeptide, expression of which is upregulated in an epithelial tumor cell; (vi) a pair of primers that, when used in a nucleic acid amplification reaction with an endothelial cell mRNA template specifically amplifies a nucleic acid encoding a human EG-1 polypeptide (SEQ ID NO:2). In certain embodiments, the nucleic acid comprises a sequence encoding an EG-1 polypeptide (e.g. SEQ ID NO:2). Certain nucleic acids comprise the nucleotide sequence of the coding region of SEQ ID NO:1. The nucleic acid sequence can comprise a vector. In certain embodiments, the nucleic acid is an EG-1 specific probe, e.g. a nucleic acid that specifically hybridizes to an EG-1 nucleic acid (e.g. gDNA, mRNA, cDNA, etc.) under stringent conditions. Preferred nucleic acids are at least 10, more preferably at least 15, and most preferably at least 20, 25, 50, or 100 nucleotides in length. The nucleic acid can, optionally, be labeled with a detectable label (e.g., a magnetic label, a radioactive label, a colorimetric label, a fluorescent label, etc.).

In another embodiment, this invention provides EG-1 polypeptides. Preferred polypeptides are encoded by an EG-1 nucleic acid (e.g. as described above) and expression of the polypeptide is upregulated in an endothelial and/or an epithelial cell. In certain embodiments expression of the polypeptide is upregulated in an epithelial cancer cell and/or a cell comprising a tissue undergoing angiogenesis. Preferred polypeptides comprise the amino acid sequence of SEQ ID NO:2 or conservative substitutions thereof.

In still another embodiment this invention provides a cell transfected with an EG-1 nucleic acid (e.g. a nucleic acid as described above) where the nucleic acid encodes an EG-1 polypeptide fragment (e.g. as illustrated in Table 1), or a full-length EG-1 polypeptide.

This invention also provides antibodies that specifically bind to an EG-1 polypeptide. Preferred antibodies specifically bind to a polypeptide comprising the amino acid sequence of SEQ ID NO:2 or a fragment thereof (e.g. a fragment comprising at lest 4, more preferably at least 8, 10, or 12, and most preferably at least 15, 18, or 20 contiguous amino acids). Preferred antibodies include, but are not limited to polyclonal antibodies, monoclonal antibodies, or single-chain antibodies.

This invention also provides a method of screening for a test agent that modulates tissue angiogenesis and/or tumorigenesis. The method involves contacting a cell (e.g. an endothelial cell, an epithelial cell, a tumor cell etc.) comprising an EG-1 gene with a test agent; and detecting a change in the expression or activity of an EG-1 gene product (e.g. EG-1 mRNA, EG-1 polypeptide, etc.) as compared to the expression or activity of a EG-1 gene product in a control cell, where a difference in the expression or activity of EG-1 in the contacted cell and the control cell indicates that the agent alters tissue angiogenesis and/or tumorigenesis. In certain embodiments, the control cell is the same type of cell contacted with the test agent at a lower concentration. In certain embodiments, the lower concentration is the absence of the test agent. The expression of the EG-1 gene product can be detected by detecting EG-1 mRNA in the sample (e.g. by hybridizing EG-1 mRNA to a probe that specifically hybridizes to an EG-1 nucleic acid). In certain embodiments EG-1 is detected by a hybridization according to a method selected from the group consisting of a Northern blot, a Southern blot using DNA derived from the EG-1 RNA, an array hybridization, an affinity chromatography, and an in situ hybridization. In certain embodiments the EG-1 specific probe is a member of a plurality of probes that forms an array of probes. In certain embodiments, the level of EG-1 mRNA is measured using a nucleic acid amplification reaction. In certain embodiments the expression of EG-1 gene product is detected by detecting the level of an EG-1 protein (or fragment thereof) in the biological sample. The EG-1 protein can be detected by a number of methods including, but not limited to capillary electrophoresis, a Western blot, mass spectroscopy, ELISA, immunochromatography, and immunohistochemistry. In certain embodiments of the assay the cell is cultured ex vivo. In certain embodiments of the assay the test agent is contacted to an animal comprising a cell containing the EG-1 nucleic acid or the EG-1 protein.

In still another embodiment this invention provides a method of prescreening for an agent that modulates tissue angiogenesis and/or tumorigenesis. The method involves i) contacting an EG-1 nucleic acid or an EG-1 protein with a test agent; and ii) detecting specific binding of the test agent to the EG-1 protein or nucleic acid where specific binding indicates that the test agent is a candidate modulator of tissue angiogenesis and/or tumorigenesis. The method can further involve recording test agents that specifically bind to said EG-1 nucleic acid or protein in a database of candidate agents that modulate tissue angiogenesis and/or tumorigenesis. In certain embodiments the test agent is not an antibody, and/or not a protein, and/or not a nucleic acid. In certain embodiments, the test agent is a small organic molecule. The detecting can comprise detecting specific binding of the test agent to said EG-1 nucleic acid (e.g. via a Northern blot, a Southern blot using DNA derived from a EG-1 RNA, an array hybridization, an affinity chromatography, an in situ hybridization, etc.). In certain embodiments the detecting can comprises detecting specific binding of the test agent to an EG-1 protein or a fragment thereof (e.g. via capillary electrophoresis, a Western blot, mass spectroscopy, ELISA, immunochromatography, immunohistochemistry, etc.). The test agent can be contacted directly to the EG-1 nucleic acid or to the EG-1 protein. The test agent can be contacted to a cell containing the EG-1 nucleic acid or the EG-1 protein. The test agent can be contacted to (administered to) an animal comprising a cell containing the EG-1 nucleic acid or the EG-1 protein.

In still another embodiment, this invention provides a transgenic (knockout) animal comprising a recombinantly modified EG-1 gene such that said recombinantly modified gene does not transcribe a functional EG-1 protein. The transgenic animal can be heterozygous or homozygous for the recombinantly modified EG-1 gene. Preferred animals are mammals including, but not limited to cattle, goats, sheep, canines, felines, largomorphs, rodents, murines, primates (especially non-human primates), pigs, and the like. Particularly preferred animals include murines (e.g. a mouse). In certain embodiments, all cells of the animal comprises the modified EG-1 gene, while in certain other embodiments, the animal is chimeric for cells comprising said recombinantly modified EG-1 gene.

This invention also provides a method for identifying a predilection to developing one or more symptoms of a disease characterized by abnormal angiogenesis. The method involves obtaining a biological sample from the organism (e.g. a human, a non-human mammal); and detecting overexpression of an EG-1 gene product. Expression or Overexpression can be assayed by a wide variety of methods (see, e.g., methods listed above).

In still another embodiment, this invention provides a method of inhibiting angiogenesis and/or tumorigenesis. The method involves inhibiting the expression or activity of an EG-1 gene product. The inhibiting can be by any of a variety of methods including, but not limited contacting an EG-1 nucleic acid with a ribozyme that specifically cleaves the EG-1 nucleic acid, contacting an EG-1 nucleic acid with a catalytic DNA that specifically cleaves the EG-1 nucleic acid, transfecting a cell comprising an EG-1 gene with a nucleic acid that inactivates the EG-1 gene by homologous recombination with the EG-1 gene, transfecting a cell comprising a with a nucleic acid encoding an intrabody that specifically binds an EG-1 polypeptide, transfecting a cell comprising an EG-1 gene with an EG-1 antisense molecule, and contacting an EG-1 polypeptide with an antibody that specifically binds the EG-1 polypeptide. In certain embodiments, the inhibiting comprises contacting an EG-1 polypeptide with an antibody that specifically binds the EG-1 polypeptide. In certain embodiments, the antibody is an antibody that specifically binds an EG-1 fragment selected from the EG-1 fragments listed in Table 1. Preferred antibodies include, but are not limited to polyclonal antibodies, monoclonal antibodies, and single-chain antibodies.

### Definitions

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

As used herein, the term “derived from a nucleic acid” (e.g., an mRNA) refers to a nucleic acid or protein nucleic acid for whose synthesis the referenced nucleic acid or a subsequence thereof has ultimately served as a template. Thus, a cDNA reverse transcribed or RT-PCR'd from an mRNA, an RNA transcribed from that cDNA, a DNA amplified from the cDNA, an RNA transcribed from the amplified DNA, etc., are all derived from the mRNA. In preferred embodiments, detection of such derived products is indicative of the presence and/or abundance of the original nucleic acid in a sample.

A “nucleic acid derived from an EG-1 gene or cDNA” refers to a nucleic acid whose synthesis the EG-1 gene or cDNA has ultimately served as a template. Thus, for example, a cDNA reverse transcribed from a EG-1 mRNA, an RNA transcribed from that cDNA, a DNA amplified from the cDNA, an RNA transcribed from the amplified DNA, etc., are all nucleic acids derived from the EG-1 gene or cDNA.

An EG-1 nucleic acid refers to a nucleic acid derived from an EG-1 gene, mRNA or cDNA, or a nucleic acid having the same sequence as a nucleic acid derived from a EG-1 gene, mRNA or cDNA. An EG-1 nucleic acid also includes fragments of such nucleic acids. In preferred embodiments, the fragments are of sufficient length to uniquely identify them as EG-1 gene mRNA or cDNA subsequences. Preferred fragments are at least 10 nucleotides, more preferably at least 15 nucleotides, still more preferably at last 20 nucleotides, and most preferably at least 25, 50, 100 or 200 nucleotides in length.

An EG-1 peptide is a peptide encoded by an EG-1 nucleic acid. Typical EG-1 peptides are typically upregulated in tissues undergoing angiogenesis and/or tumorigenesis.

The term “antibody”, as used herein, includes various forms of modified or altered antibodies, such as an intact immunoglobulin, an Fv fragment containing only the light and heavy chain variable regions, an Fv fragment linked by a disulfide bond (Brinkmann et al. (1993) Proc. Natl. Acad. Sci. USA, 90: 547-551), an Fab or (Fab)′2 fragment containing the variable regions and parts of the constant regions, a single-chain antibody and the like (Bird et al. (1988) Science 242: 424-426; Huston et al. (1988) Proc. Nat. Acad. Sci. USA 85: 5879-5883). The antibody may be of animal (especially mouse or rat) or human origin or may be chimeric (Morrison et al. (1984) Proc Nat. Acad. Sci. USA 81: 6851-6855) or humanized (Jones et al. (1986) Nature 321: 522-525, and published UK patent application #8707252).

The terms “binding partner”, or “capture agent”, or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, etc.), refers to a binding reaction which is determinative of the presence of a biomolecule in a heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all, to other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, N.Y. (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions, e.g., containing formamide (see, e.g., Sambrook (1989) Molecular Cloning: A Laboratory Manual (2nd ed.) Vol. 1-3, Cold Spring Harbor Laboratory, Cold Spring Harbor Press, N.Y., and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

The term “conservative substitution” is used in reference to proteins or peptides to reflect amino acid substitutions that do not substantially alter the activity (specificity or binding affinity) of the molecule. Typically conservative amino acid substitutions involve substitution one amino acid for another amino acid with similar chemical properties (e.g. charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

### BRIEF DESCRIPTION OF THE DRAWINGS

FIGS. 1A and 1B show alignments of EG-1. FIG. 1A shows the alignment of deduced amino-acid sequence (SEQ ID NO:2) of Endothelial-derived gene-1 (EG-1) with its nucleotide sequence (SEQ ID NO:1). FIG. 1B shows the alignment of deduced amino-acid sequence of Endothelial-derived gene-1 (EG-1) (SEQ ID NO:1) with its murine (SEQ ID NO:3) and Drosophila (SEQ ID NO:4) counterparts.

FIGS. 2A and 2B show features of the Endothelial-derived gene (EG-1) gene. FIG. 2A shows a hydrophilicity plot of Endothelial-derived gene-1 (EG-1). FIG. 2B shows structural features of Endothelial-derived gene-1 (EG-1). Motif analysis reveals a proline-rich region (#13-39), one N-glycosylation site (# 66-69), four casein kinase II phosphorylation sites (# 43-46, 50-68-71, 75-78), and two N-myristoylation sites (# 6-11, 76-81). There is some alignment with the following: Tim 10/DDP (deafness dystonia protein) family zinc finger (aa # 29-97), poly A polymerase regulatory subunit (aa # 77-87), interleukin-8 like small cytokines (intecrine/chemokine) (aa # 125-136), and regulatory subunit of type II PKA (cAMP-dependent protein kinase) R-subunit (aa # 137-167).

FIGS. 3A and 3B show induction of endothelial derived gene (EG-1) expression by tumor conditioned media. FIG. 3A: Induction of endothelial-derived gene EG-1 expression by tumor conditioned media in sparse conditions (50% confluency). Control HUVECs (human umbilical vein endothelial cells) were cultured in growth media (lane 1), starved in plain DMEM (lane 2), and stimulated HUVECs in tumor conditioned media (lane 3). Twenty μg of RNA was hybridized with EG-1 and β-actin cDNA probes. FIG. 3B: Induction of endothelial-derived gene-1 (EG-1) expression by tumor conditioned media in confluent conditions (90-100% confluency). Control HUVECs (human umbilical vein endothelial cells) were cultured in growth media (lane 1), starved in plain DMEM (lane 2), and stimulated HUVECs in tumor conditioned media (lane 3). Twenty μg of RNA was hybridized with EG-1 and β-actin cDNA probes.

FIG. 4 illustrates induction of endothelial-derived gene-1 (EG-1) expression by bFGF (basic fibroblast growth factor) and TNFα (tumor necrosis factor alpha). HUVECs (human umbilical vein endothelial cells) were cultured in EGM (endothelial growth media) or plain DMEM. Confluent condition is 80% confluency (lane 1), and sparse condition is <50% confluency (lanes 2-3). Sparse HUVECs were exposed to 5 ng/ml bFGF (lane 4) or 200 units/ml TNFα (lane 5). Twenty μg of RNA was hybridized with EG-1 and β-actin cDNA probes.

FIG. 5 illustrates the presence of endothelial-derived gene-1 (EG-1) in different types of endothelial cells. Twenty μg of RNA from HUVECs (human umbilical vein endothelial cells), human aortic endothelial cells (HAECs), and human microvascular endothelial cells (HUVECs) were hybridized with EG1 and β-actin cDNA probes. Control cells were cultured in EGM (endothelial growth media) (lanes 1, 4, and 7). Starved cells were cultured in plain DMEM (lanes 2, 5, and 8). Conditioned media from the malignant melanoma C8161 was used to stimulate endothelial cells (lanes 3, 6, and 9).

FIG. 6 illustrates the presence of endothelial-derived gene-1 (EG-1) in different types of human tissues. mRNA multi-tissue blots from Origene were hybridized with EG-1 and β-actin cDNA probes.

FIG. 7 illustrates the expression of endothelial-derived gene-1 (EG-1) in non-endothelial cell types. Twenty μg of RNA from HUVECs (human umbilical vein endothelial cells) and other cell lines were hybridized with EG-1 and β-actin cDNA probes. Benign human cells include liver, lung, myoepithelial HMS, and fibroblast Ccd-sk-27. Malignant human cells include melanoma C8161, prostate cancer LnCap, colon cancer Colo-205, and breast cancer T47D and Mda-Mb-231.

FIG. 8 illustrates the presence of endothelial-derived gene-1 (EG-1) in the endothelial cells of capillaries (panel A), arteries (panel B), veins (panel C), spleen endotheliocytes (panel D), placenta Hoffbauer cells (panel E), and hemangioma blood vessels (panel F). In situ hybridization was performed as detailed in Example 1.

FIG. 9 illustrates the presence of Endothelial-derived gene-1 (EG-1) in normal breast (panel A), breast cancer (panel B), normal colon (panel C), colon cancer (panel D), normal prostate (panel E), prostate cancer (panel F), normal lung (panel G), and lung cancer (panel H). In situ hybridization was performed as detailed in Example 1.

FIG. 10 shows an illustration of EG-1 structure with peptide an antibody positions as identified in Table 1.

FIG. 11 illustrates inhibition of HUVEC proliferation by peptide 10.

FIG. 12 illustrates apoptosis of HUVEC mediated by antibody 10.

FIG. 13 illustrates inhibition of HUVEC migration by anti-EG-1 antibodies (ab-1 through ab-5).

FIG. 14 illustrates inhibition of HUVEC tube formation by anti-EG-1 antibodies (ab-1 through ab-5).

FIGS. 15A, 15B, 15C, 15D, and 15E illustrate inhibition of HUVEC adhesion to breast cancer MDA-MB-231 cells. *P<0.05. FIG. 15A: Antibody Ab-1; FIG. 15B: Antibody Ab-2; FIG. 15C: Antibody Ab-3; FIG. 15D: Antibody Ab-4; FIG. 15E: Antibody Ab-5.

FIGS. 16A, 16B, 16C, and 16D illustrate inhibition of HUVEC adhesion to colon cancer colo-205 cells. *P<0.05.

### DETAILED DESCRIPTION

This invention pertains to the identification, characterization and isolation of a novel endothelial-derived gene. The gene was identified by suppression subtractive hybridization (SSH) on control human umbilical vein endothelial cells (HUVECs) versus HUVECs exposed to tumor-conditioned media. We found that a novel cDNA (Genbank accession # AF358829, SEQ ID NO:5) is differentially expressed in endothelial cells on Northern analysis, and named it endothelial-derived gene-1 (EG-1).

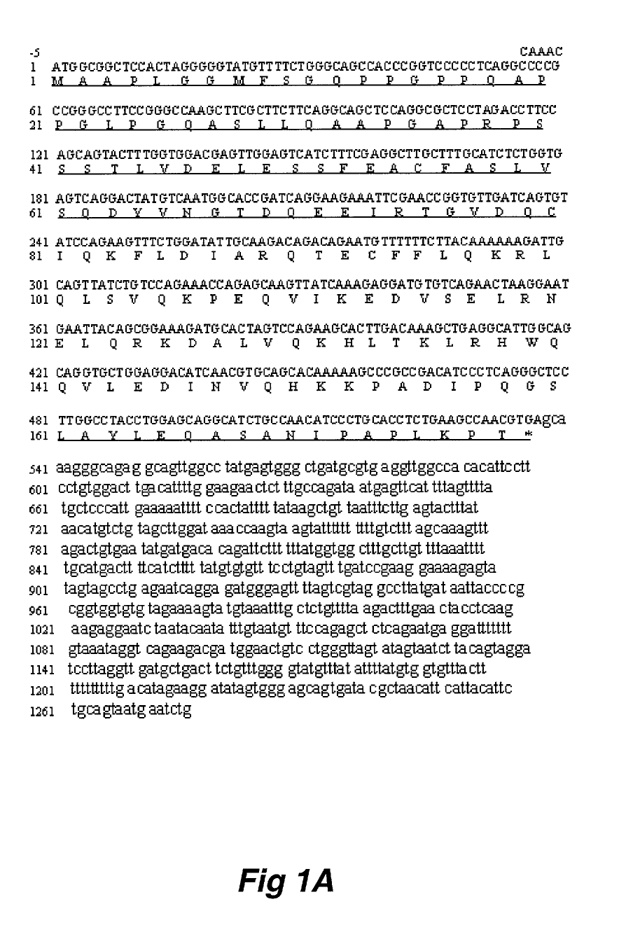

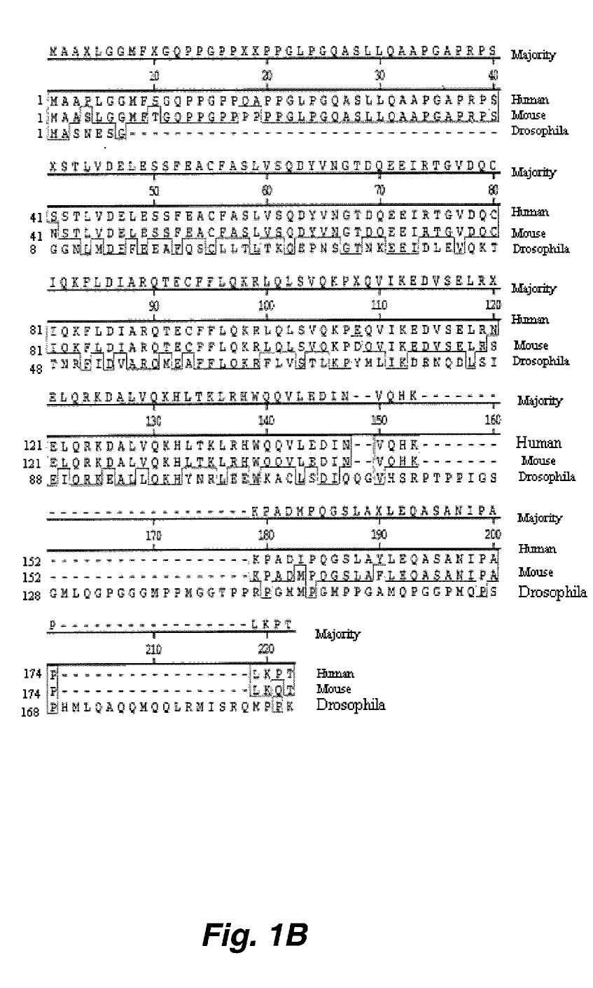

The gene product is predicted to encode a 178-aa, 19.5 kD protein, and is localized to chromosome 4. Human EG-1 has significant homology to a mouse cDNA (94%, FIG. 1B) and a to a Drosophila cDNA (31%, FIG. 1B). On Northern analysis, endothelial cells express two EG-1 RNA species (1.2 kb and 2.4 kb). The expression of either transcript is upregulated by endothelial cells when exposed to tumor conditioned media.

Transcripts are present abundantly in highly vascular tissues such as placenta, testis, and liver. Both Northern analysis and in situ hybridization studies show that this gene is expressed in other cell types as well, predominantly the epithelial type. Breast cancer, prostate cancer, and colon cancer cells show elevated expression of the higher 2.4 kb RNA form. Our data suggest that EG-1 is associated with a stimulated state in endothelial and epithelial cells, without being bound to a particular theory, we believe it has a role in tumor angiogenesis.

Consequently, EG-1 makes a good target to screen for agents that modulate (upregulate or downregulate) tissue angiogenesis and/or tumorigenesis. In certain embodiments, a cell, tissue, or organism is contacted/administered a test agent and the cell, tissue or organism is screened for upregulation or downregulation of an EG-1 gene product where the upregulation or downregulation indicates that the test agent is a good candidate modulator for tissue angiogenesis and/or tumorigenesis.

EG-1 is also a good target (marker) for the presence of a cancer cell and/or as a prognostic for the outcome of a cancer. Upregulation o EG-1 expression is believed to be associated with malignant transformation. In addition, cells overexpressing EG-1 are expected to show a predisposition to participate in angiogenic events and hence to more readily form solid tumors. Detection of EG-1 upregulation can therefore provide an indicator of the presence of a cancer and/or the likelihood/prognosis of tumor formation.

It is also believed that EG-1 provides a good therapeutic target for the treatment of various cancers or other pathologies characterized by abnormal angiogenesis. The therapeutic moieties can be selected to inhibit or to upregulate EG-1 activity or, EG-1 can be used as a target to specifically direct EG-1 targeted therapeutics to cells overexpressing EG-1.

In addition to various assays and therapeutic methods, the EG-1 nucleic acids can be used to prepare probes, e.g., to detect and/or quantify EG-1 expression. The EG-1 polypeptides can be expressed and used to screen for anti-EG-1 antibodies that are also useful for detecting EG-1 expression. Thus, in various embodiments, this invention provides EG-1 nucleic acids, EG-1 polypeptides, cells transfected with EG-1 nucleic acids and capable of expressing heterologous EG-1 polypeptides, methods of screening for modulators of tissue angiogenesis and/or tumorigenesis, and the like.

I. EG-1 Nucleic Acids.

A) Preparation of EG-1 Nucleic Acids.

In certain embodiments, this invention provides novel EG-1 nucleic acids. A human EG-1 cDNA (coding region of SEQ ID NO:1) and the predicted amino acid sequence (SEQ ID NO:2) are illustrated in FIG. 1.

Using the information provided herein, (e.g. EG-1 cDNA sequence, primers, etc.) the nucleic acids (e.g., encoding full length EG-1, or subsequences of the EG-1 cDNA, genomic DNA, mRNA, etc) are prepared using standard methods well known to those of skill in the art. For example, the EG-1 nucleic acid(s) may be cloned, or amplified by in vitro methods, such as the polymerase chain reaction (PCR), the ligase chain reaction (LCR), the transcription-based amplification system (TAS), the self-sustained sequence replication system (SSR), etc. A wide variety of cloning and in vitro amplification methodologies are well known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, N.Y., (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science, 241: 1077-1080; Van Brunt (1990) Biotechnology, 8: 291-284: Wu and Wallace, (1989) Gene, 4: 560; and Barringer et al. (1990) Gene, 89: 117.

The isolation and expression of an EG-1 nucleic acid is illustrated in Example 1. In one preferred embodiment, the EG-1 cDNA can be isolated by routine cloning methods. The cDNA sequence provided in SEQ ID NO:1 can be used to provide probes that specifically hybridize to the EG-1 gene, in a genomic DNA sample, or to the EG-1 mRNA, in a total RNA sample (e.g., in a Southern blot). Once the target EG-1 nucleic acid is identified (e.g., in a Southern blot), it can be isolated according to standard methods known to those of skill in the art (see, e.g., Sambrook et al. (1989) Molecular Cloning: A Laboratory Manual, 2nd Ed., Vols. 1-3, Cold Spring Harbor Laboratory; Berger and Kimmel (1987) Methods in Enzymology, Vol. 152: Guide to Molecular Cloning Techniques, San Diego: Academic Press, Inc.; or Ausubel et al. (1987) Current Protocols in Molecular Biology, Greene Publishing and Wiley-Interscience, New York). Methods of screening human tissue samples for EG-1 are provided in Example 1.

In certain embodiment, the human EG-1 cDNA can be isolated by amplification methods such as polymerase chain reaction (PCR). For example, the EG-1 sequence is amplified from a cDNA sample (e.g., double stranded placental cDNA (Clontech)) using the primers routinely derived from the sequence illustrated in FIG. 1 (SEQ. ID NO:1). Illustrative primers include, but are not limited to 3′ Primer 1 (TCA CGT TGG CTT CAG AGG, SEQ ID NO:7) and 5′ Primer 2 (ATG GCG GCT CCA CTA GGG, SEQ ID NO:8), or 3′ Primer 3 (TCA CGT TGG CTT CAG AGG, SEQ ID NO:9) and 5′ Primer 4 (CAC CAT GGC GGC TCC ACT AGG G, SEQ ID NO:10) for convenient insertion into an expression vector. Typical amplification conditions include 30 cycles of 1 minute denaturing at 94° C., 1 minute annealing at 54° C., 3 minutes of extension at 72° C., followed by a final 15 minute extension at 72° C. Typical template includes, but is not limited to reverse-transcribed DNA from an endothelial cell.

B) Labeling of EG-1 Nucleic Acids.

Particularly where the EG-1 gDNA, cDNA, mRNA or their subsequences are to be used as nucleic acid probes, it is often desirable to label the nucleic acids with detectable labels. The labels can be incorporated by any of a number of means well known to those of skill in the art. In certain embodiments, the label is simultaneously incorporated during an amplification step in the preparation of the EG-1 nucleic acids. Thus, for example, polymerase chain reaction (PCR) with labeled primers or labeled nucleotides will provide a labeled amplification product. In another embodiment, transcription amplification using a labeled nucleotide (e.g. fluoresceine-labeled UTP and/or CTP) incorporates a label into the transcribed nucleic acids.

In certain embodiments, a label can be added directly to an original nucleic acid sample (e.g., mRNA, polyA mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore). Suitable labels are described below.

The label may be added to nucleic acid(s) prior to, or after use (e.g. prior to or after hybridization). So called “direct labels” are detectable labels that are directly attached to or incorporated into the target (sample) nucleic acid prior to use. In contrast, so called “indirect labels” are joined to the nucleic acid after use (e.g. hybridization). Often, the indirect label is attached to a binding moiety that has been attached to the target nucleic acid prior to a hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Tijssen (1993) Laboratory Techniques in Biochemistry and Molecular Biology, Vol. 24: Hybridization With Nucleic Acid Probes, Elsevier, N.Y.

The labels can be attached directly or through a linker moiety. In general, the site of label or linker-label attachment is not limited to any specific position. For example, a label can be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties

Detectable labels suitable for use in the present invention include any composition detectable by spectroscopic, photochemical, biochemical, immunochemical, electrical, optical or chemical means. Useful labels in the present invention include, but are not limited to, biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold (e.g., gold particles in the 40 -80 nm diameter size range scatter green light with high efficiency) or colored glass or plastic (e.g., polystyrene, polypropylene, latex, etc.) beads. Patents teaching the use of such labels includ U.S. Pat. Nos. 3,817,837; 3,850,752; 3,939,350; 3,996,345; 4,277,437; 4,275,149; and 4,366,241.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Illustrative spin labels include, but are not limited to nitroxide free radicals.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

II. Cloning and Expression of EG-1.

It is often desirable to provide isolated EG-1 polyepeptides. These polypeptides can be used to raise an immune response and thereby generate antibodies specific to the intact EG-1 or to various subsequences or domains thereof. As explained below, EG-1 polypeptides and various fragments thereof can be conveniently produced using synthetic chemical syntheses or recombinant expression methodologies. In addition to the intact full-length EG-1 polypeptide, in some embodiments, it is often desirably to express immunogenically relevant fragments (e.g. fragments that can be used to raise specific anti-EG-1 antibodies).

A) De novo Chemical Synthesis.

The EG-1 polypeptide(s), or fragments thereof can be synthesized using standard chemical peptide synthesis techniques. Where the desired subsequences are relatively short (e.g., when a particular antigenic determinant is desired) the molecule can be synthesized as a single contiguous polypeptide. Where larger molecules are desired, subsequences can be synthesized separately (in one or more units) and then fused by condensation of the amino terminus of one molecule with the carboxyl terminus of the other molecule thereby forming a peptide bond.

Solid phase synthesis in which the C-terminal amino acid of the sequence is attached to an insoluble support followed by sequential addition of the remaining amino acids in the sequence is the preferred method for the chemical synthesis of the polypeptides of this invention. Techniques for solid phase synthesis are described by Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. (1963) J. Am. Chem. Soc., 85: 2149-2156, and Stewart et al. (1984) Solid Phase Peptide Synthesis, 2nd ed. Pierce Chem. Co., Rockford, Ill.

B) Recombinant Expression.

In a certain embodiments, the EG-1 proteins or subsequences thereof, are synthesized using recombinant expression systems. Generally this involves creating a DNA sequence that encodes the desired protein, placing the DNA in an expression cassette under the control of a particular promoter, expressing the protein in a host, isolating the expressed protein and, if required, renaturing the protein.

DNA encoding the EG-1 proteins described herein can be prepared by any suitable method as described above, including, for example, cloning and restriction of appropriate sequences or direct chemical synthesis.

This nucleic acid can be easily ligated into an appropriate vector containing appropriate expression control sequences (e.g. promoter, enhancer, etc.), and, optionally, containing one or more selectable markers (e.g. antibiotic resistance genes).

The nucleic acid sequences encoding EG-1 proteins or protein subsequences can be expressed in a variety of host cells, including, but not limited to, E. coli, other bacterial hosts, yeast, fungus, and various higher eukaryotic cells such as insect cells (e.g. SF3), the COS, CHO and HeLa cells lines and myeloma cell lines. The recombinant protein gene will be operably linked to appropriate expression control sequences for each host. For E. coli this can include a promoter such as the T7, trp, or lambda promoters, a ribosome binding site and preferably a transcription termination signal. For eukaryotic cells, the control sequences can include a promoter and often an enhancer (e.g., an enhancer derived from immunoglobulin genes, SV40, cytomegalovirus, etc.), and a polyadenylation sequence, and may include splice donor and acceptor sequences.

The plasmids of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant EG-1 protein(s) can be purified according to standard procedures of the art, including ammonium sulfate precipitation, affinity columns, column chromatography, gel electrophoresis and the like (see, generally, R. Scopes, (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred. Once purified, partially or to homogeneity as desired, the polypeptides may then be used (e.g., as immunogens for antibody production). The cloning and expression of a EG-1 polypeptides is illustrated in Example 1.

One of skill in the art would recognize that after chemical synthesis, biological expression, or purification, the EG-1 protein(s) may possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (see, e.g., Debinski et al. (1993) J. Biol. Chem., 268: 14065-14070; Kreitman and Pastan (1993) Bioconjug. Chem., 4: 581-585; and Buchner, et al., (1992) Anal. Biochem., 263-270). Debinski et al., for example, describes the denaturation and reduction of inclusion body proteins in guanidine-DTE. The protein is then refolded in a redox buffer containing oxidized glutathione and L-arginine.

One of skill would recognize that modifications can be made to the EG-1 proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

III. Assays for Modulators of EG-1.

As indicated above, in one aspect, this invention pertains to the discovery that EG-1 is implicated in tissue angiogenesis and/or tumorigenesis. EG-1 thus provides a target to screen for modulators of tissue angiogenesis or tumorigenesis, or of screening for cancers and/or evaluating the severity of a cancer and/or the likelihood of metastatic cells being present and/or developing and/or evaluating the prognosis of a cancer. The methods involve detecting the expression level and/or activity level of an EG-1 or an EG-1 gene product. Elevated levels indicate increased angiogenic activity and/or potential and indicate the presence of a cancer cell, a proclivity for tumorigenesis, or a proclivity for other pathologies characterized by abnormal angiogenesis.

In diagnostic/prognostic applications EG-1 expression need not be dispositive with respect to the existence of a particular pathology. Rather, EG-1 expression level is used in the context of a differential diagnosis for that pathology. Accordingly, upregulation of EG-1 is used along with a number of other factors to provide a definitive diagnosis. In this context, EG-1 expression level is simply an indicator (one of many possible indicators) of a particular pathology (e.g. abnormal angiogenesis, tumorigenesis, etc).

Similarly, when screening for modulators, a positive assay result need not indicate the particular test agent is a good pharmaceutical. Rather a positive result can simply indicate that the test agent can be used to modulate EG-1 activity and/or can also serve as a lead compound in the development of other modulators.

Using the nucleic acid sequences and/or amino acid sequences provided herein EG-1 copy number and/or, EG-1 expression level, and/or EG-1 activity level can be directly measured according to a number of different methods as described below. In particular, expression levels of a gene can be altered by changes in the copy number of the gene, and/or by changes in the transcription of the gene product (i.e. transcription of mRNA), and/or by changes in translation of the gene product (i.e. translation of the protein), and/or by post-translational modification(s) (e.g. protein folding, glycosylation, etc.). Thus useful assays of this invention include assaying for copy number, level of transcribed mRNA, level of translated protein, activity of translated protein, etc. Examples of such approaches are described below.

### A) Nucleic-acid Based Assays

1) Target Molecules.

Changes in expression level can be detected by measuring changes in mRNA and/or a nucleic acid derived from the mRNA (e.g. reverse-transcribed cDNA, etc.). In order to measure the EG-1 expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism, or from cells in culture. The sample may be of any biological tissue or fluid. Biological samples may also include organs or sections of tissues such as frozen sections taken for histological purposes.

The nucleic acid (e.g., mRNA nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g, Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of EG-1 in a sample, the nucleic acid sample is one in which the concentration of the EG-1 mRNA transcript(s), or the concentration of the nucleic acids derived from the EG-1 mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, the EG-1-containing nucleic acid sample is the total mRNA or a total cDNA isolated and/or otherwise derived from a biological sample. The nucleic acid may be isolated from the sample according to any of a number of methods well known to those of skill in the art as indicated above.

2) Hybridization-based Assays.

Using the EG-1 sequences provided herein (see, e.g., SEQ ID NO:1) detecting and/or quantifying the EG-1 transcript(s) can be routinely accomplished using nucleic acid hybridization techniques (see, e.g., Sambrook et al. supra). For example, one method for evaluating the presence, absence, or quantity of EG-1 reverse-transcribed cDNA involves a “Southern Blot”. In a Southern Blot, the DNA (e.g., reverse-transcribed EG-1 mRNA), typically fragmented and separated on an electrophoretic gel, is hybridized to a probe specific for EG-1 (or to a mutant thereof). Comparison of the intensity of the hybridization signal from the EG-1 probe with a “control” probe (e.g. a probe for a “housekeeping gene) provides an estimate of the relative expression level of the target nucleic acid.

Alternatively, the EG-1 mRNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target EG-1 mRNA. Appropriate controls (e.g. probes to housekeeping genes) provide a reference for evaluating relative expression level.

An alternative means for determining the EG-1 expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non-specific hybridization.

3) Amplification-based Assays.

In another embodiment, amplification-based assays can be used to measure EG-1 expression (transcription) level. In such amplification-based assays, the target nucleic acid sequences (i.e., EG-1) act as template(s) in amplification reaction(s) (e.g. Polymerase Chain Reaction (PCR) or reverse-transcription PCR (RT-PCR)). In a quantitative amplification, the amount of amplification product will be proportional to the amount of template (e.g., EG-1 mRNA) in the original sample. Comparison to appropriate (e.g. healthy tissue or cells unexposed to the test agent) controls provides a measure of the EG-1 transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One typical internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The known nucleic acid sequence(s) for EG-1 are sufficient to enable one of skill to routinely select primers to amplify any portion of the gene.

4) Hybridization Formats and Optimization of Hybridization Conditions.

a) Array-based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high-density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high-density arrays is also described in U.S. Pat. Nos. 5,744,305, 5,800,992 and 5,445,934.

b) Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemi-luminescent agents, enzymes, and antibodies that can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.).

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and Detection of Nucleic Acids.

The probes used herein for detection of EG-1 expression levels can be full length or less than the full length of the EG-1 or mutants thereo. Shorter probes are empirically tested for specificity. Preferred probes are sufficiently long so as to specifically hybridize with the EG-1 target nucleic acid(s) under stringent conditions. The preferred size range is from about 10, 15, or 20 bases to the length of the EG-1 mRNA, more preferably from about 30 bases to the length of the EG-1 mRNA, and most preferably from about 40 bases to the length of the EG-1 mRNA. The probes are typically labeled, with a detectable label as described above.

### B) Polypeptide-based Assays

1) Assay Formats.

In addition to, or in alternative to, the detection of EG-1 nucleic acid expression level(s), alterations in expression of EG-1 can be detected and/or quantified by detecting and/or quantifying the amount and/or activity of translated EG-1 polypeptide.

2) Detection of Expressed Protein

The polypeptide(s) encoded by the EG-1 gene can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the EG-1 polypeptide(s) are detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using labeled antibodies (e.g., labeled sheep anti-mouse antibodies) that specifically bind to a domain of the antibody.

In preferred embodiments, the EG-1 polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (EG-1 polypeptide). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Typical immunoassays for detecting the target polypeptide(s) are either competitive or noncompetitive. Noncompetitive immunoassays are assays in which the amount of captured analyte is directly measured. In one “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (EG-1 polypeptide) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind EG-1 polypeptide(s), either alone or in combination. In the case where the antibody that binds EG-1 polypeptide is not labeled, a different detectable marker, for example, an enzyme-labeled antibody capable of binding to the monoclonal antibody which binds the EG-1 polypeptide, may be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention may also be other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds EG-1 polypeptides is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents may also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein can be routinely produced as described below.

3) Antibodies to EG-1 Polypeptides.

Either polyclonal or monoclonal antibodies can be used in the immunoassays of the invention described herein. Polyclonal antibodies are typically raised by multiple injections (e.g. subcutaneous or intramuscular injections) of substantially pure polypeptides or antigenic polypeptides into a suitable non-human mammal. The antigenicity of the target peptides can be determined by conventional techniques to determine the magnitude of the antibody response of an animal that has been immunized with the peptide. Generally, the peptides that are used to raise antibodies for use in the methods of this invention should generally be those which induce production of high titers of antibody with relatively high affinity for target polypeptides encoded by EG-1 or variants thereof.

If desired, the immunizing peptide can be coupled to a carrier protein by conjugation using techniques that are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit).

The antibodies are then obtained from blood samples taken from the mammal. The techniques used to develop polyclonal antibodies are known in the art (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, for example, Coligan, et al. (1991) Unit 9, Current Protocols in Immunology, Wiley Interscience).

In certain embodiments, however, the antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)^{2′}, and/or single-chain antibodies (e.g. scFv) which are capable of binding an epitopic determinant. Also, in this context, the term “mab's of the invention” refers to monoclonal antibodies with specificity for a polypeptide encoded by EG-1.

The general method used for production of hybridomas secreting mAbs is well known (Kohler and Milstein (1975) Nature, 256:495). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines. Confirmation of specificity among mAb's can be accomplished using relatively routine screening techniques (such as the enzyme-linked immunosorbent assay, or “ELISA”) to determine the elementary reaction pattern of the mAb of interest.

Antibody fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20 fold-1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V_{H} and V_{L} repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1:M to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

It will also be recognized that antibodies can be prepared by any of a number of commercial services (e.g., Berkeley antibody laboratories, Bethyl Laboratories, Anawa, Eurogenetec, etc.).

### C) Assay Optimization

The assays of this invention have immediate utility in screening for agents that modulate the EG-1 expression and/or activity in a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration. Conversely, where it is desired to screen for modulators that alter transcription the EG-1 gene, nucleic acid based assays are preferred.

Routine selection and optimization of assay formats is well known to those of ordinary skill in the art.

### D) Pre-screening for Agents that Bind EG-1 or EG-1 Polypeptide

In certain embodiments it is desired to pre-screen test agents for the ability to interact with (e.g. specifically bind to) an EG-1 (or mutant/allele) nucleic acid or polypeptide. Specifically, binding test agents are more likely to interact with and thereby modulate EG-1 expression and/or activity. Thus, in some preferred embodiments, the test agent(s) are pre-screened for binding to EG-1 nucleic acids or to EG-1 proteins before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the EG-1 protein or nucleic acid is immobilized and exposed to a test agent (which can be labeled), or alternatively, the test agent(s) are immobilized and exposed to an EG-1 protein or to a EG-1 nucleic acid (which can be labeled). The immobilized moiety is then washed to remove any unbound material and the bound test agent or bound EG-1 nucleic acid or protein is detected (e.g. by detection of a label attached to the bound molecule). The amount of immobilized label is proportional to the degree of binding between the EG-1 protein or nucleic acid and the test agent.

### E) Scoring the Assay(s)

The assays of this invention are scored according to standard methods well known to those of skill in the art. The assays of this invention are typically scored as positive where there is a difference between the activity seen with the test agent present or where the test agent has been previously applied, and the (usually negative) control. In preferred embodiments, the change is a statistically significant change, e.g. as determined using any statistical test suited for the data set provided (e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.). Preferably the statistically significant change is significant at least at the 85%, more preferably at least at the 90%, still more preferably at least at the 95%, and most preferably at least at the 98% or 99% confidence level). In certain embodiments, the change is at least a 10% change, preferably at least a 20% change, more preferably at least a 50% change and most preferably at least a 90% change.

### F) Agents for Screening: Combinatorial Libraries (e.g., Small Organic Molecules)

Virtually any agent can be screened according to the methods of this invention. Such agents include, but are not limited to nucleic acids, proteins, sugars, polysaccharides, glycoproteins, lipids, and small organic molecules. The term small organic molecules typically refers to molecules of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays, as described herein to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library such as a polypeptide (e.g., mutein) library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, Dec. 26, 1991), encoded peptides (PCT Publication WO 93/20242, Oct. 14, 1993), random bio-oligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include, but are not limited to, automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist and the Venture™ platform, an ultra-high-throughput synthesizer that can run between 576 and 9,600 simultaneous reactions from start to finish (see Advanced ChemTech, Inc. Louisville, Ky.)). Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo, ChemStar, Ltd, Moscow, RU, 3D Pharmaceuticals, Exton, Pa., Martek Biosciences, Columbia, Md, etc.).

### G) High Throughout Screening

Any of the assays described herein are amenable to high-throughput screening (HTS). Moreover, the cells utilized in the methods of this invention need not be contacted with a single test agent at a time. To the contrary, to facilitate high-throughput screening, a single cell may be contacted by at least two, preferably by at least 5, more preferably by at least 10, and most preferably by at least 20 test compounds. If the cell scores positive, it can be subsequently tested with a subset of the test agents until the agents having the activity are identified.

High throughput assays for hybridizaiton assays, immunoassays, and for various reporter gene products are well known to those of skill in the art. For example, multi-well fluorimeters are commercially available (e.g., from Perkin-Elmer).

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

### H) Modulator Databases

In certain embodiments, the agents that score positively in the assays described herein (e.g. show an ability to modulate EG-1 expression) can be entered into a database of putative and/or actual modulators of EG-1 expression and/or tissue angiogenesis or tumorigenesis. The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

IV. Diagnostics/prognostics.

The assays described above, can also be used in diagnostic/prognostic applications. EG-1 provides an effective marker for the detection/diagnosis of a wide variety of cancers particularly cancers of urogenital tissues. Diagnosis of disease or risk of disease based on measured levels of EG-1 can be made by comparison to levels measured in a disease-free control group or background levels measured in a particular patient. The diagnosis can be confirmed by correlation of the assay results with other signs of disease known to those skilled in the clinical arts, such as the diagnostic standards for breast cancer, gastric cancer, prostate cancer, etc.

The levels of EG-1 that are indicative of the development or amelioration of a particular cancer by disease and, to a lesser extent, by patient. Appropriate background EG-1 levels in particular tissues, pathologies, and patients or patient populations or control populations can be determined by routine screening according to standard methods well known to those of skill in the art.

For purposes of diagnosing the onset, progression, or amelioration of disease, variations in the levels of EG-1 of interest will be those which differ by a statistically significant level from the normal (i.e., healthy) population or from the level measured in the same individual at a different time, and which correlate to other clinical signs of disease occurrence and/or prognosis and/or amelioration known to those skilled in the clinical art pertaining to the disease of interest.

Thus, in general, any diagnosis or prognosis indicated by EG-1 measurements made according to the methods of the invention will be independently confirmed with reference to clinical manifestations of disease known to practitioners of ordinary skill in the clinical arts.

In prognostic applications, EG-1 levels are evaluated to estimate the risk of progression of a cancer or the risk of recurrence of a cancer and thereby provide information that facilitates the selection of treatment regimen. Without being bound to a particular theory, it is believed that tissues or tumors are heterogeneous (even within a particular tissue type or tumor type, e.g. colorectal cancer) with respect to elevated expression of EG-1. Those tissues showing elevated expression of EG-1 also show a high likelihood of tumorigenesis or where tumorigenesis has occurred, disease progression. Thus, measurement of EG-1 levels (before, during [i.e. in tissues removed during surgery], or after primary tumor removal) provides a prognostic indication of the likelihood of tumor recurrence. Where pathologies show elevated EG-1 levels (e.g. as compared to those in normal healthy subjects) more aggressive adjunct therapies (e.g. chemotherapy and/or radiotherapy) may be indicated.

V. EG-1-targeted Therapeutics.

In certain embodiments, this invention contemplates the use of EG-1 targeted therapeutics in the treatment of cancers or other pathologies characterized by abnormal angiogenesis. Typically such methods will entail administration of an agent that modulates (e.g. downregulates) EG-1 transcription, translation, or activity. Such agents include, but are not limited to agents identified according to the screening methods described herein.

Other agents can also be used to downregulate expression of EG-1. Such agents include, but are not limited to antisense molecules, EG-1 specific riibozymes, EG-1 specific catalytic DNAs, EG-1-specific RNAi, intrabodies directed against EG-1 proteins, and “gene therapy” approaches that knock out EG-1.

### A) Antisense Approaches

EG-1 gene expression can be downregulated or entirely inhibited by the use of antisense molecules. An “antisense sequence or antisense nucleic acid” is a nucleic acid that is complementary to the coding EG-1 mRNA nucleic acid sequence or a subsequence thereof. Binding of the antisense molecule to the EG-1 mRNA interferes with normal translation of the EG-1 polypeptide.

Thus, in accordance with certain embodiments of this invention, antisense molecules include oligonucleotides and oligonucleotide analogs that are hybridizable with EG-1 messenger RNA. This relationship is commonly denominated as “antisense.” The oligonucleotides and oligonucleotide analogs are able to inhibit the function of the RNA, either its translation into protein, its translocation into the cytoplasm, or any other activity necessary to its overall biological function. The failure of the messenger RNA to perform all or part of its function results in a reduction or complete inhibition of expression of EG-1 polypeptides.

In the context of this invention, the term “oligonucleotide” refers to a polynucleotide formed from naturally-occurring bases and/or cyclofuranosyl groups joined by native phosphodiester bonds. This term effectively refers to naturally-occurring species or synthetic species formed from naturally-occurring subunits or their close homologs. The term “oligonucleotide” may also refer to moieties which function similarly to oligonucleotides, but which have non naturally-occuring portions. Thus, oligonucleotides may have altered sugar moieties or inter-sugar linkages. Exemplary among these are the phosphorothioate and other sulfur containing species that are known for use in the art. In accordance with some preferred embodiments, at least one of the phosphodiester bonds of the oligonucleotide has been substituted with a structure which functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention.

In one embodiment, the internucleotide phosphodiester linkage is replaced with a peptide linkage. Such peptide nucleic acids tend to show improved stability, penetrate the cell more easily, and show enhances affinity for their target. Methods of making peptide nucleic acids are known to those of skill in the art (see, e.g., U.S. Pat. Nos: 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCH₃, OCN, O(CH₂)[n]NH₂ or O(CH₂)[n]CH₃, where n is from 1 to about 10, and other substituents having similar properties.

Such oligonucleotides are best described as being functionally interchangeable with natural oligonucleotides or synthesized oligonucleotides along natural lines, but which have one or more differences from natural structure. All such analogs are comprehended by this invention so long as they function effectively to hybridize with messenger RNA of EG-1 to inhibit the function of that RNA.

The oligonucleotides in accordance with certain embodiments of this invention comprise from about 3 to about 50 subunits. It is more preferred that such oligonucleotides and analogs comprise from about 8 to about 25 subunits and still more preferred to have from about 12 to about 20 subunits. As will be appreciated, a subunit is a base and sugar combination suitably bound to adjacent subunits through phosphodiester or other bonds. The oligonucleotides used in accordance with this invention can be conveniently and routinely made through the well-known technique of solid phase synthesis. Equipment for such syntheses is sold by several vendors (e.g. Applied Biosystems). Any other means for such synthesis may also be employed, however, the actual synthesis of the oligonucleotides is well within the talents of the routineer. It is also will known to prepare other oligonucleotide such as phosphorothioates and alkylated derivatives.

### B) Catalytic RNAs and DNAs

1) Ribozymes.

In another approach, EG-1 expression can be inhibited by the use of ribozymes. As used herein, “ribozymes” include RNA molecules that contain antisense sequences for specific recognition, and an RNA-cleaving enzymatic activity. The catalytic strand cleaves a specific site in a target (EG-1) RNA, preferably at greater than stoichiometric concentration. Two “types” of ribozymes are particularly useful in this invention, the hammerhead ribozyme (Rossi et al. (1991) Pharmac. Ther. 50: 245-254) and The hairpin ribozyme (Hampel et al. (1990) Nucl. Acids Res. 18: 299-304, and U.S. Pat. No. 5,254,678).

Because both hammerhead and hairpin ribozymes are catalytic molecules having antisense and endoribonucleotidase activity, ribozyme technology has emerged as a potentially powerful extension of the antisense approach to gene inactivation. The ribozymes of the invention typically consist of RNA, but such ribozymes may also be composed of nucleic acid molecules comprising chimeric nucleic acid sequences (such as DNA/RNA sequences) and/or nucleic acid analogs (e.g., phosphorothioates).

Accordingly, within one aspect of the present invention ribozymes have the ability to inhibit EG-1 expression. Such ribozymes may be in the form of a “hammerhead” (for example, as described by Forster and Symons (1987) Cell 48: 211-220,; Haseloff and Gerlach (1988) Nature 328: 596-600; Walbot and Bruening (1988) Nature 334: 196; Haseloff and Gerlach (1988) Nature 334: 585) or a “hairpin” (see, e.g. U.S. Pat. No. 5,254,678 and Hampel et al., European Patent Publication No. 0 360 257, published Mar. 26, 1990), and have the ability to specifically target, cleave and EG-1 nucleic acids.

The ribozymes for this invention, as well as DNA encoding such ribozymes and other suitable nucleic acid molecules can be chemically synthesized using methods well known in the art for the synthesis of nucleic acid molecules. Alternatively, Promega, Madison, Wis., USA, provides a series of protocols suitable for the production of RNA molecules such as ribozymes. The ribozymes also can be prepared from a DNA molecule or other nucleic acid molecule (which, upon transcription, yields an RNA molecule) operably linked to an RNA polymerase promoter, e.g., the promoter for T7 RNA polymerase or SP6 RNA polymerase. Such a construct may be referred to as a vector. Accordingly, also provided by this invention are nucleic acid molecules, e.g., DNA or cDNA, coding for the ribozymes of this invention. When the vector also contains an RNA polymerase promoter operably linked to the DNA molecule, the ribozyme can be produced in vitro upon incubation with the RNA polymerase and appropriate nucleotides. In a separate embodiment, the DNA may be inserted into an expression cassette (see, e.g., Cotten and Birnstiel (1989) EMBO J 8(12):3861-3866; Hempel et al. (1989) Biochem. 28: 4929-4933, etc.).

After synthesis, the ribozyme can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

The ribozyme molecule also can be in a host prokaryotic or eukaryotic cell in culture or in the cells of an organism/patient. Appropriate prokaryotic and eukaryotic cells can be transfected with an appropriate transfer vector containing the DNA molecule encoding a ribozyme of this invention. Alternatively, the ribozyme molecule, including nucleic acid molecules encoding the ribozyme, may be introduced into the host cell using traditional methods such as transformation using calcium phosphate precipitation (Dubensky et al. (1984) Proc. Natl. Acad. Sci., USA, 81: 7529-7533), direct microinjection of such nucleic acid molecules into intact target cells (Acsadi et al. (1991) Nature 352: 815-818), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al. (1990) Proc. Natl. Acad. Sci., USA, 89: 6094), lipofection (Felgner et al. (1989) Proc. Natl. Acad. Sci. USA 84: 7413-7417), microprojectile bombardment (Williams et al. (1991) Proc. Natl. Acad. Sci., USA, 88: 2726-2730), polycation compounds such as polylysine, receptor specific ligands, liposomes entrapping the nucleic acid molecules, spheroplast fusion whereby E coli containing the nucleic acid molecules are stripped of their outer cell walls and fused to animal cells using polyethylene glycol, viral transduction, (Cline et al., (1985) Pharmac. Ther. 29: 69; and Friedmann et al. (1989) Science 244: 1275), and DNA ligand (Wu et al (1989) J. Biol. Chem. 264: 16985-16987), as well as psoralen inactivated viruses such as Sendai or Adenovirus. In one preferred embodiment, the ribozyme is introduced into the host cell utilizing a lipid, a liposome or a retroviral vector.

When the DNA molecule is operatively linked to a promoter for RNA transcription, the RNA can be produced in the host cell when the host cell is grown under suitable conditions favoring transcription of the DNA molecule. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6):1202-1207, 1993; Zabner et al. (1993) Cell 75(2): 207-216; Li et al. (1993) Hum Gene Ther. 4(4): 403-409; Caillaud et al. (1993) Eur. J Neurosci. 5(10): 1287-1291), adeno-associated vector type 1 (“AAV-1”) or adeno-associated vector type 2 (“AAV-2”) (see WO 95/13365; Flotte et al. (1993) Proc. Natl. Acad. Sci., USA, 90(22):10613-10617), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y., and Kreigler (1990) Gene Transfer and Expression: A Laboratory Manual, W.H. Freeman and Company, New York.

To produce ribozymes in vivo utilizing vectors, the nucleotide sequences coding for ribozymes are preferably placed under the control of a strong promoter such as the lac, SV40 late, SV40 early, or lambda promoters. Ribozymes are then produced directly from the transfer vector in vivo

2) Catalytic DNA

In a manner analogous to ribozymes, DNAs are also capable of demonstrating catalytic (e.g. nuclease) activity. While no such naturally-occurring DNAs are known, highly catalytic species have been developed by directed evolution and selection. Beginning with a population of 10¹⁴ DNAs containing 50 random nucleotides, successive rounds of selective amplification, enriched for individuals that best promote the Pb²⁺-dependent cleavage of a target ribonucleoside 3′-O-P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ (see, e.g., Breaker and Joyce (1994) Chem Biol 4: 223-229.

In later work, using a similar strategy, a DNA enzyme was made that could cleave almost any targeted RNA substrate under simulated physiological conditions. The enzyme is comprised of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10⁹ M⁻¹min⁻¹ under multiple turnover conditions, exceeding that of any other known nucleic acid enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to EG-1 mRNA thereby acting like a ribozyme.

### C) RNAi Inhibition of EG-1 Expression

Post-transcriptional gene silencing (PTGS) or RNA interference (RNAi) refers to a mechanism by which double-stranded (sense strand) RNA (dsRNA) specifically blocks expression of its homologous gene when injected, or otherwise introduced into cells. The discovery of this incidence came with the observation that injection of antisense or sense RNA strands into Caenorhabditis elegans cells resulted in gene-specific inactivation (Guo and Kempheus (1995) Cell 81: 611-620). While gene inactivation by the antisense strand was expected, gene silencing by the sense strand came as a surprise. Adding to the surprise was the finding that this gene-specific inactivation actually came from trace amounts of cantaminating dsRNA (Fire et al. (1998) Nature 391: 806-811).

Since then, this mode of post-transcriptional gene silencing has been tied to a wide variety of organisms: plants, flies, trypanosomes, planaria, hydra, zebrafish, and mice (Zamore et al. (2000). Cell 101: 25-33; Gura (2000) Nature 404: 804-808). RNAi activity has been associated with functions as disparate as transposon-silencing, anti-viral defense mechanisms, and gene regulation (Grant (1999) Cell 96: 303-306).

By injecting dsRNA into tissues, one can inactivate specific genes not only in those tissues, but also during various stages of development. This is in contrast to tissue-specific knockouts or tissue-specific dominant-negative gene expressions, which do not allow for gene silencing during various stages of the developmental process (Gura (2000) Nature 404: 804-808). The double-stranded RNA is cut by a nuclease activity into 21-23 nucleotide fragments. These fragments, in turn, target the homologous region of their corresponding mRNA, hybridize, and result in a double-stranded substrate for a nuclease that degrades it into fragments of the same size (Hammond et al. (2000) Nature, 404: 293-298; Zamore et al. (2000). Cell 101: 25-33).

Double stranded RNA (dsRNA) can be introduced into cells by any of a wide variety of means. Such methods include, but are not limited to lipid-mediated transfection (e.g. using reagents such as lipofectamine), liposome delivery, dendrimer-mediated transfection, and gene transfer using a viral or bacterial vector. Where the vector expresses (transcribes) a single-stranded RNA, the vector can be designed to trasnscribe two complementary RNA strands that will then hybridize to form a double-stranded RNA.

### D) Knocking out EG-1

In another approach, EG-1 can be inhibited/downregulated simply by “knocking out” the gene. Typically this is accomplished by disrupting the EG-1 gene, the promoter regulating the gene or sequences between the promoter and the gene. Such disruption can be specifically directed to EG-1 by homologous recombination where a “knockout construct” contains flanking sequences complementary to the domain to which the construct is targeted. Insertion of the knockout construct (e.g. into the EG-1 gene) results in disruption of that gene. The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, the cell and its progeny will no longer express the gene or will express it at a decreased level, as the DNA is now disrupted by the antibiotic resistance gene.

Knockout constructs can be produced by standard methods known to those of skill in the art. The knockout construct can be chemically synthesized or assembled, e.g., using recombinant DNA methods. The DNA sequence to be used in producing the knockout construct is digested with a particular restriction enzyme selected to cut at a location(s) such that a new DNA sequence encoding a marker gene can be inserted in the proper position within this DNA sequence. The proper position for marker gene insertion is that which will serve to prevent expression of the native gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon). Preferably, the enzyme selected for cutting the DNA will generate a longer arm and a shorter arm, where the shorter arm is at least about 300 base pairs (bp). In some cases, it will be desirable to actually remove a portion or even all of one or more exons of the gene to be suppressed so as to keep the length of the knockout construct comparable to the original genomic sequence when the marker gene is inserted in the knockout construct. In these cases, the genomic DNA is cut with appropriate restriction endonucleases such that a fragment of the proper size can be removed.

The marker gene can be any nucleic acid sequence that is detectable and/or assayable, however typically it is an antibiotic resistance gene or other gene whose expression or presence in the genome can easily be detected. The marker gene is usually operably linked to its own promoter or to another strong promoter from any source that will be active or can easily be activated in the cell into which it is inserted; however, the marker gene need not have its own promoter attached as it may be transcribed using the promoter of the gene to be suppressed. In addition, the marker gene will normally have a polyA sequence attached to the 3′ end of the gene; this sequence serves to terminate transcription of the gene. Preferred marker genes are any antibiotic resistance gene including, but not limited to neo (the neomycin resistance gene) and beta-gal (beta-galactosidase).

After the genomic DNA sequence has been digested with the appropriate restriction enzymes, the marker gene sequence is ligated into the genomic DNA sequence. using methods well known to the skilled artisan (see, e.g., Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, N.Y.; and Current Protocols in Molecular Biology, F. M. Ausubel et al., eds., Current Protocols, a joint venture between Greene Publishing Associates, Inc. and John Wiley & Sons, Inc., (1994) Supplement). The ends of the DNA fragments to be ligated must be compatible; this is achieved by either cutting all fragments with enzymes that generate compatible ends, or by blunting the ends prior to ligation. Blunting is done using methods well known in the art, such as for example by the use of Klenow fragment (DNA polymerase I) to fill in sticky ends.

Suitable knockout constructs have been made and used to produce EG-1 knockout mice (see, Examples herein). The knockout constructs can be delivered to cells in vivo using gene therapy delivery vehicles (e.g. retroviruses, liposomes, lipids, dendrimers, etc.) as described below. Methods of knocking out genes are well described in the literature and essentially routine to those of skill in the art (see, e.g., Thomas et al. (1986) Cell 44(3): 419-428; Thomas, et al. (1987) Cell 51(3): 503-512)1; Jasin and Berg (1988) Genes & Development 2: 1353-1363; Mansour, et al. (1988) Nature 336: 348-352; Brinster, et al. (1989) Proc Natl Acad Sci 86: 7087-7091; Capecchi (1989) Trensa in Genetics 5(3): 70-76; Frohman and Martin (1989) Cell 56: 145-147; Hasty, et al. (1991) Mol Cell Bio 11(11): 5586-5591; Jeannotte, et al. (1991) Mol Cell Biol. 11(11): 557814 5585; and Mortensen, et al. (1992) Mol Cell Biol. 12(5): 2391-2395.

The use of homologous recombination to alter expression of endogenous genes is also described in detail in U.S. Pat. No. 5,272,071, WO 91/09955, WO 93/09222, WO 96/29411, WO 95/31560, and WO 91/12650.

Production of the knockout animals of this invention is not dependent on the availability of ES cells. In various embodiments, knockout animals of this invention can be produced using methods of somatic cell nuclear transfer. In preferred embodiments using such an approach, a somatic cell is obtained from the species in which the EG-1 gene is to be knocked out. The cell is transfected with a construct that introduces a disruption in the EG-1 gene (e.g. via heterologous recombination) as described herein. Cells harboring a knocked out EG-1 gene are selected as described herein. The nucleus of such cells harboring the knockout is then placed in an unfertilized enucleated egg (e.g., eggs from which the natural nuclei have been removed by microsurgery). Once the transfer is complete, the recipient eggs contained a complete set of genes, just as they would if they had been fertilized by sperm. The eggs are then cultured for a period before being implanted into a host mammal (of the same species that provided the egg) where they are carried to term, culminating in the berth of a transgenic animal comprising a nucleic acid construct containing one or more disrupted Ttpa genes (e.g. the disrupted Ttpa gene).

The production of viable cloned mammals following nuclear transfer of cultured somatic cells has been reported for a wide variety of species including, but not limited to frogs (McKinnell (1962) J. Hered. 53, 199-207), calves (Kato et al. (1998) Science 262: 2095-2098), sheep (Cambell et al. (1996) Nature 380: 64-66), mice (Wakayamaand Yanagimachi (1999) Nat. Genet. 22: 127-128), goats (Baguisi et al. (1999) Nat. Biotechnol. 17: 456-461), monkeys (Meng et al. (1997) Biol. Reprod. 57: 454-459), and pigs (Bishop et al. (2000) Nature Biotechnology 18: 1055-1059). Nuclear transfer methods have also been used to produce clones of transgenic animals. Thus, for example, the production of transgenic goats carrying the human antithrobin III gene by somatic cell nuclear transfer has been reported (Baguisi et al. (1999) Nature Biotechnology 17: 456-461).

Using methods of nuclear transfer as describe in these and other references, cell nuclei derived from differentiated fetal or adult, mammalian cells are transplanted into enucleated mammalian oocytes of the same species as the donor nuclei. The nuclei are reprogrammed to direct the development of cloned embryos, which can then be transferred into recipient females to produce fetuses and offspring, or used to produce cultured inner cell mass (CICM) cells. The cloned embryos can also be combined with fertilized embryos to produce chimeric embryos, fetuses and/or offspring.

Somatic cell nuclear transfer also allows simplification of transgenic procedures by working with a differentiated cell source that can be clonally propagated. This eliminates the need to maintain the cells in an undifferentiated state, thus, genetic modifications, both random integration and gene targeting, are more easily accomplished. Also by combining nuclear transfer with the ability to modify and select for these cells in vitro, this procedure is more efficient than previous transgenic embryo techniques.

Nuclear transfer techniques or nuclear transplantation techniques are known in the literature. See, in particular, Campbell et al. (1995) Theriogenology, 43:181; Collas et al. (1994) Mol. Report Dev., 38:264-267; Keefer et al. (1994) Biol. Reprod., 50:935-939; Sims et al. (1993) Proc. Natl. Acad. Sci., USA, 90:6143-6147; WO 94/26884; WO 94/24274, WO 90/03432, U.S. Pat. Nos. 5,945,577, 4,944,384, 5,057,420 and the like.

Having shown that disruption of the EG-1 gene produces a high-growth (hg) phenotype, and that hg animals are viable, one of skill will recognize that there are a wide number of animals including natural and transgenic animals that have other desirable phenotypes and that can be used to practice the invention by use of ES cells and/or somatic nuclear transfer. Preferred animals are mammals including, but not limited to porcine, cows, cattle, goats, sheep, canines, felines, largomorphs, rodents, murines, primates (especially non-human primates), and the like.

### E) Intrabodies

In still another embodiment, EG-1 expression/activity can be inhibited by transfecting the subject cell(s) (e.g., cells of the vascular endothelium) with a nucleic acid construct that expresses an intrabody. An intrabody is an intracellular antibody, in this case, capable of recognizing and binding to a EG-1 polypeptide. The intrabody is expressed by an “antibody cassette”, containing a sufficient number of nucleotides coding for the portion of an antibody capable of binding to the target (EG-1 polypeptide) operably linked to a promoter that will permit expression of the antibody in the cell(s) of interest. The construct encoding the intrabody is delivered to the cell where the antibody is expressed intracellularly and binds to the target EG-1, thereby disrupting the target from its normal action. This antibody is sometimes referred to as an “intrabody”.

In one preferred embodiment, the “intrabody gene” (antibody) of the antibody cassette would utilize a cDNA, encoding heavy chain variable (V_{H}) and light chain variable (V_{L}) domains of an antibody which can be connected at the DNA level by an appropriate oligonucleotide as a bridge of the two variable domains, which on translation, form a single peptide (referred to as a single chain variable fragment, “sFv”) capable of binding to a target such as an EG-1 protein. The intrabody gene preferably does not encode an operable secretory sequence and thus the expressed antibody remains within the cell.

Anti-EG-1 antibodies suitable for use/expression as intrabodies in the methods of this invention can be readily produced by a variety of methods. Such methods include, but are not limited to, traditional methods of raising “whole” polyclonal antibodies, which can be modified to form single chain antibodies, or screening of, e.g. phage display libraries to select for antibodies showing high specificity and/or avidity for EG-1. Such screening methods are described above in some detail.

The antibody cassette is delivered to the cell by any of the known means. One preferred delivery system is described in U.S. Pat. No. 6,004,940. Methods of making and using intrabodies are described in detail in U.S. Pat. Nos. 6,072,036, 6,004,940, and 5,965,371.

### F) Small Organic Molecules

In still another embodiment, EG-1 expression and/or EG-1 protein activity can be inhibited by the use of small organic molecules. Such molecules include, but are not limited to molecules that specifically bind to the DNA comprising the EG-1 promoter and/or coding region, molecules that bind to and complex with EG-1 mRNA, molecules that inhibit the signaling pathway that results in EG-1 upregulation, and molecules that bind to and/or compete with EG-1 polypeptides. Small organic molecules effective at inhibiting EG-1 expression can be identified with routine screening using the methods described herein.

The methods of inhibiting EG-1 expression described above are meant to be illustrative and not limiting. In view of the teachings provided herein, other methods of inhibiting EG-1 will be known to those of skill in the art.

### G) Modes of Administration

The mode of administration of the EG-1 blocking agent depends on the nature of the particular agent. Antisense molecules, catalytic RNAs (ribozymes), catalytic DNAs, small organic molecules, RNAi, and other molecules (e.g. lipids, antibodies, etc.) used as EG-1 inhibitors may be formulated as pharmaceuticals (e.g. with suitable excipient) and delivered using standard pharmaceutical formulation and delivery methods as described below. Antisense molecules, catalytic RNAs (ribozymes), catalytic DNAs, and additionally, knockout constructs, and constructs encoding intrabodies can be delivered and (if necessary) expressed in target cells (e.g. vascular endothelial cells) using methods of gene therapy, e.g. as described below.

1) “Pharmaceutical” Formulations.

In order to carry out the methods of the invention, one or more inhibitors of EG-1 expression (e.g. ribozymes, antibodies, antisense molecules, small organic molecules, etc.) are administered to a cell, tissue, or organism, to induce a high growth (hg) phenotype. Various inhibitors may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

The EG-1 inhibitors and various derivatives and/or formulations thereof are useful for parenteral, topical, oral, or local administration, such as by aerosol or transdermally, for prophylactic and/or therapeutic treatment of undergrowth disorders or overgrowth disorders, such as cases of uncontrolled cell proliferation which are the causal factor in tumor development. The pharmaceutical compositions can be administered in a variety of unit dosage forms depending upon the method of administration. Suitable unit dosage forms, include, but are not limited to powders, tablets, pills, capsules, lozenges, suppositories, implants etc.

The EG-1 inhibitors and various derivatives and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

The concentration of active agent(s) in the formulation can vary widely, and will be selected primarily based on fluid volumes, viscosities, body weight and the like in accordance with the particular mode of administration selected and the patient's needs. Typically, the active agent(s) are administered in an amount sufficient to alter expression of EG-1, i.e., an “effective amount”. Single or multiple administrations of the compositions may be administered depending on the dosage and frequency as required and tolerated by the organism or cell or tissue system. In any event, the composition should provide a sufficient quantity of the active agents of this invention to effectively alter EG-1 expression and preferably to induce or reduce an hg phenotype.

2) “Genetic” Delivery Methods.

As indicated above, antisense molecules, catalytic RNAs (ribozymes), catalytic DNAs, RNAi, and additionally, knockout constructs, and constructs encoding intrabodies can be delivered and transcribed and/or expressed in target cells (e.g. vascular endothelial cells) using methods of gene therapy. Thus, in certain preferred embodiments, the nucleic acids encoding knockout constructs, intrabodies, antisense molecules, catalytic RNAs or DNAs, etc. are cloned into gene therapy vectors that are competent to transfect cells (such as human or other mammalian cells) in vitro and/or in vivo.

Many approaches for introducing nucleic acids into cells in vivo, ex vivo and in vitro are known. These include lipid or liposome based gene delivery (WO 96/18372; WO 93/24640; Mannino and Gould-Fogerite (1988) BioTechniques 6(7): 682-691; Rose U.S. Pat. No. 5,279,833; WO 91/06309; and Felgner et al. (1987) Proc. Natl. Acad. Sci. USA 84: 7413-7414) and replication-defective retroviral vectors harboring a therapeutic polynucleotide sequence as part of the retroviral genome (see, e.g., Miller et al. (1990) Mol. Cell. Biol. 10:4239 (1990); Kolberg (1992) J. NIH Res. 4: 43, and Cornetta et al. (1991) Hum. Gene Ther. 2: 215).

For a review of gene therapy procedures, see, e.g., Anderson, Science (1992) 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Böhm (eds) Springer-Verlag, Heidelberg Germany; and Yu et al., (1994) Gene Therapy, 1:13-26.

Widely used vector systems include, but are not limited to adenovirus, adeno associated virus, and various retroviral expression systems. The use of adenoviral vectors is well known to those of skill and is described in detail, e.g., in WO 96/25507. Particularly preferred adenoviral vectors are described by Wills et al. (1994) Hum. Gene Therap. 5: 1079-1088.

Adeno-associated virus (AAV)-based vectors used to transduce cells with target nucleic acids, e.g., in the in vitro production of nucleic acids and peptides, and in in vivo and ex vivo gene therapy procedures are describe, for example, by West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like). Other suitable viral vectors include, but are not limited to herpes virus, lentivirus, and vaccinia virus.

Alone, or in combination with viral vectors, a number of non-viral vectors are also useful for transfecting cells to express constructs that block or inhibit EG-1 expression. Suitable non-viral vectors include, but are not limited to, plasmids, cosmids, phagemids, liposomes, water-oil emulsions, polethylene imines, biolistic pellets/beads, and dendrimers.

Liposomes were first described in 1965 as a model of cellular membranes and quickly were applied to the delivery of substances to cells. Liposomes entrap DNA by one of two mechanisms which has resulted in their classification as either cationic liposomes or pH-sensitive liposomes. Cationic liposomes are positively charged liposomes which interact with the negatively charged DNA molecules to form a stable complex. Cationic liposomes typically consist of a positively charged lipid and a co-lipid. Commonly used co-lipids include dioleoyl phosphatidylethanolamine (DOPE) or dioleoyl phosphatidylcholine (DOPC). Co-lipids, also called helper lipids, are in most cases required for stabilization of liposome complex. A variety of positively charged lipid formulations are commercially available and many other are under development. Two of the most frequently cited cationic lipids are lipofectamine and lipofectin. Lipofectin is a commercially available cationic lipid first reported by Phil Felgner in 1987 to deliver genes to cells in culture. Lipofectin is a mixture of N-[1-(2, 3-dioleyloyx)propyl]-N-N-N-trimethyl ammonia chloride (DOTMA) and DOPE.

DNA and lipofectin or lipofectamine interact spontaneously to form complexes that have a 100% loading efficiency. In other words, essentially all of the DNA is complexed with the lipid, provided enough lipid is available. It is assumed that the negative charge of the DNA molecule interacts with the positively charged groups of the DOTMA. The lipid:DNA ratio and overall lipid concentrations used in forming these complexes are extremely important for efficient gene transfer and vary with application. Lipofectin has been used to deliver linear DNA, plasmid DNA, and RNA to a variety of cells in culture. Shortly after its introduction, it was shown that lipofectin could be used to deliver genes in vivo. Following intravenous administration of lipofectin-DNA complexes, both the lung and liver showed marked affinity for uptake of these complexes and transgene expression. Injection of these complexes into other tissues has had varying results and, for the most part, are much less efficient than lipofectin-mediated gene transfer into either the lung or the liver.

PH-sensitive, or negatively-charged liposomes, entrap DNA rather than complex with it. Since both the DNA and the lipid are similarly charged, repulsion rather than complex formation occurs. Yet, some DNA does manage to get entrapped within the aqueous interior of these liposomes. In some cases, these liposomes are destabilized by low pH and hence the term pH- sensitive. To date, cationic liposomes have been much more efficient at gene delivery both in vivo and in vitro than pH-sensitive liposomes. pH-sensitive liposomes have the potential to be much more efficient at in vivo DNA delivery than their cationic counterparts and should be able to do so with reduced toxicity and interference from serum protein.

In another approach dendrimers complexed to the DNA have been used to transfect cells. Such dendrimers include, but are not limited to, “starburst” dendrimers and various dendrimer polycations.

Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. These dendrimers may be prepared as disclosed in PCT/US83/02052, and U.S. Pat. Nos. 4,507,466, 4,558,120, 4,568,737, 4,587,329, 4,631,337, 4,694,064, 4,713,975, 4,737,550, 4,871,779, 4,857,599.

Typically, the dendrimer polycations comprise a core molecule upon which polymers are added. The polymers may be oligomers or polymers which comprise terminal groups capable of acquiring a positive charge. Suitable core molecules comprise at least two reactive residues which can be utilized for the binding of the core molecule to the oligomers and/or polymers. Examples of the reactive residues are hydroxyl, ester, amino, imino, imido, halide, carboxyl, carboxyhalide maleimide, dithiopyridyl, and sulfhydryl, among others. Preferred core molecules are ammonia, tris-(2-aminoethyl)amine, lysine, ornithine, pentaerythritol and ethylenediamine, among others. Combinations of these residues are also suitable as are other reactive residues.

Oligomers and polymers suitable for the preparation of the dendrimer polycations of the invention are pharmaceutically-acceptable oligomers and/or polymers that are well accepted in the body. Examples of these are polyamidoamines derived from the reaction of an alkyl ester of an α,β-ethylenically unsaturated carboxylic acid or an α,β-ethylenically unsaturated amide and an alkylene polyamine or a polyalkylene polyamine, among others. Preferred are methyl acrylate and ethylenediamine. The polymer is preferably covalently bound to the core molecule.

The terminal groups that may be attached to the oligomers and/or polymers should be capable of acquiring a positive charge. Examples of these are azoles and primary, secondary, tertiary and quaternary aliphatic and aromatic amines and azoles, which may be substituted with S or O, guanidinium, and combinations thereof. The terminal cationic groups are preferably attached in a covalent manner to the oligomers and/or polymers. Preferred terminal cationic groups are amines and guanidinium. However, others may also be utilized. The terminal cationic groups may be present in a proportion of about 10 to 100% of all terminal groups of the oligomer and/or polymer, and more preferably about 50 to 100%.

The dendrimer polycation may also comprise 0 to about 90% terminal reactive residues other than the cationic groups. Suitable terminal reactive residues other than the terminal cationic groups are hydroxyl, cyano, carboxyl, sulfhydryl, amide and thioether, among others, and combinations thereof. However others may also be utilized.

The dendrimer polycation is generally and preferably non-covalently associated with the polynucleotide. This permits an easy disassociation or disassembling of the composition once it is delivered into the cell. Typical dendrimer polycation suitable for use herein have a molecular weight ranging from about 2,000 to 1,000,000 Da, and more preferably about 5,000 to 500,000 Da. However, other molecule weights are also suitable. Preferred dendrimer polycations have a hydrodynamic radius of about 11 to 60 Å., and more preferably about 15 to 55 Å. Other sizes, however, are also suitable. Methods for the preparation and use of dendrimers in gene therapy are well known to those of skill in the art and describe in detail, for example, in U.S. Pat. No. 5,661,025.

Where appropriate, two or more types of vectors can be used together. For example, a plasmid vector may be used in conjunction with liposomes. In the case of non-viral vectors, nucleic acid may be incorporated into the non-viral vectors by any suitable means known in the art. For plasmids, this typically involves ligating the construct into a suitable restriction site. For vectors such as liposomes, water-oil emulsions, polyethylene amines and dendrimers, the vector and construct may be associated by mixing under suitable conditions known in the art.

VI. Kits for Assaying EG-1 Activity.

In still another embodiment, this invention provides kits for assaying EG-1 copy number, and/or expression level. In certain embodiments, the kits comprise an EG-1 nucleic acid specific probe and/or an EG-1 specific antibody. The probe or antibody can, optimally, be labeled with a detectable label, the kit can include a label for such labeling.

The kits can include instructional materials providing protocols for the assays disclosed herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Identification of a Novel Endothelial-Derived Gene Eg-1

The identification of novel endothelial-derived genes is important in the study of angiogenesis, and may have potential uses in cancer diagnosis and treatment. We performed SSH (suppression subtractive hybridization) on control HUVECs (human umbilical vein endothelial cells) versus HUVECs exposed to tumor-conditioned media. We found that a novel cDNA (Genbank accession # AF358829) is differentially expressed in endothelial cells on Northern analysis, and named it Endothelial-derived gene-1 (EG-1). This gene product is predicted to encode a 178-aa, 19.5 kD protein, and is localized to chromosome #4. It has some homology to a mouse cDNA (94%) and a Drosophila cDNA (31%). On Northern analysis, endothelial cells express two EG-1 RNA species (1.2 kb and 2.4 kb). The expression of either transcript is upregulated by endothelial cells when exposed to tumor conditioned media. This phenomenon is observed only in sparse conditions (50% confluency). Transcripts are present abundantly in highly vascular tissues such as placenta, testis, and liver. Interestingly, both Northern analysis and in situ hybridization studies show that this gene is expressed in other cell types as well, predominantly the epithelial type. Breast cancer, prostate cancer, and colon cancer cells show elevated expression of the higher 2.4 kb RNA form. Our data indicate that EG-1 is associated with a stimulated state in endothelial and epithelial cells, and we believe it has a role in tumor angiogenesis.

Materials and Methods.

Sequence Analysis

The sequence of all clones was determined in both directions by automated cycle-sequencing by the UCLA Jonsson Comprehensive Cancer Center sequencing facility. Sequence analysis was performed with the Lasergene Navigator (DNASTAR, Inc., Madison, Wis.) software package and with searches of the Genbank database using BLASTN. For motif analysis, the following internet websites were used: http://pfam.wustl.edu/hmmsearch.shtml, and http://www.isrec.isb-sib.ch/software/PFSCAN form.html.

Cloning of EG-1

The full length cDNA sequence was obtained by standard molecular methods (10) using a HUVEC cDNA library. Briefly, the library was screened for desired clones using the partial fragment derived from SSH (Genbank accession # AW73573 1). The identity of the clones was validated by sequencing.

Cell Culture

Human umbilical vein endothelial cells (HUVECs) were purchased from Clonetics (San Diego, Calif.). The cells were plated on tissue culture flasks coated with 1.5% gelatin (Difco, Detroit, Mich.) and were maintained in endothelial growth media (EGM: endothelial cell growth medium completed with 10 ng/ml HEGF (human epithelial growth factor), 2% fetal calf serum (FCS, Gemini, Calabasas, Calif.), 1.0 μg/ml hydrocortisone, gentamicin and amphotericin-B (Clonetics). Human aortic endothelial cells (HAECs) and human microvascular endothelial cells (HMVECs) were purchased from Cascade (Portland, Oreg.). For some experiments, cells were rendered quiescent by “starving” in culture in Dulbecco's minimal essential medium (DMEM, Life Technologies, Carlsbad, Calif.) lacking additional supplements. For experiments with specific angiogenic factors, the endothelial cells were grown in DMEM with either bFGF (basic fibroblast growth factor, Chemicon International Inc., Temecula, Calif.) at 5 ng/ml or TNF-α (tumor necrosis factor alpha, Alexis Corp., San Diego, Calif.) at 200 units/ml.

The human melanoma line C8161 and the human breast cancer cell line Mda-Mb-231 (from American Tissue Type Culture Collection, Rockville, Md.) were maintained on non-gelatinized flasks with DMEM and 10% heat-inactivated FCS, 100 units/ml penicillin, and 100 μg/ml streptomycin (Life Technologies). The tumor conditioned media was prepared with confluent cultures of either C8161 or Mda-Mb-231 as previously described (Nguyen et al. (1997) Am. J. Path. 150: 1307-1310). Briefly, the serum-free DMEM media bathing the tumor cells over 48 hours was collected, spun, and the supernatant concentrated approximately 5-10-fold with Centripreps with a 3,000 m.w. cutoff.

Other cells used in this study included benign human fibroblast Ccd-sk-27, benign human liver, benign human lung, human breast cancer Mcf-7 and T47D, human colon cancer Colo-205 and Ls-174t, and human prostate cancer LnCap from ATCC. Human myoepithelial HMS cells were obtained from Dr. Barsky. These cells were all grown in DMEM with 10% FCS, with the exception of HMS which was grown in keratinocyte serum-free medium (K-SFM) supplemented with 50 μg/ml bovine pituitary extract and 5 ng/ml recombinant human epidermal growth factor (GIBCO/BRL, Carlsbad, Calif.).

Northern Analysis

The multi-tissue mRNA blots were purchased from Origene (Rockville, Md.). For other blots, total RNA was extracted from cell lines using Trizol™ (GIBCO/BRL). Twenty μg of total RNA was loaded per lane and resolved on 1.2% agarose gels prior to transfer to nitrocellulose membranes, as previously described (Wang et al. (2000) Microvasc. Res. 59: 394-397). The EG-1 cDNA probe was labeled by the random primer method (Feinberg and Vogelstein (1983) Anal. Biochem. 132: 6-13). All blots were also reprobed for β-actin (GIBCO/BRL) content to verify RNA quantity. Bands for Northern blots were quantitated using a Molecular Dynamic Laser Densitometer (Model PSD1) and an Image Quant Version. 1 software program.

Human Tissue

Human tissue samples were obtained from the UCLA Human Tissue Research Center. Only archival tissue was used, and the identity of the human subjects was removed so as to make the samples untracable. As for all studies involving human tissue, this study was conducted in compliance with the rules of the UCLA Human Subject Protection Committee.

In situ Hybridization.

Formalin-fixed, paraffin-embedded tissues were sectioned, placed on 3-aminopropyltriethoxysilane-treated slides (GIBCO/BRL), then baked at 60° C. for one hour. The paraffin was removed by incubation in xylene, followed by 100% ethanol. The sections were digested with 40 μg/ml of proteinase K (GIBCO/BRL) for ten minutes at 37° C., then washed with PBS. All samples were then fixed for one minute in 10% buffered formalin, washed with PBS, dehydrated through graded alcohols, and air dried in preparation for hybridization. The probe was labeled with biotin by nick translation according to the manufacturers' instructions (BioPRIME DNA Labeling System, Life Technologies). Unincorporated nucleotides were removed by column chromatography using BioGel® P-60 gels (Bio-Rad, Hercules, Calif.). Double strand probes were heat denatured for five minutes at 100° C. prior to hybridization. Hybridization was conducted using the GIBCO BRL In Situ Hybridization and Detection System. Slides were hybridized for overnight at 42° C. After hybridization, the slides were washed in 0.2×SSC. The signal was detected using streptavidin alkaline phosphatase conjugate and NBT-BCIP (nitroblue tetrazolium, 4-bromo-5-chloro-3-indolylphosphate) substrates. The slides were counterstained with Methyl Green (Sigma, St. Louis, Mo), dehydrated through graded alcohols, and mounted with Permount® solution (Fisher Scientific, Tustin, Calif.). Photography was carried out with a Leica DMLS microscope (McBain Instruments, Chatsworth, Calif.) and a Nikon N6006 camera (Tokyo, Japan).

Furthermore, for a standard fee, in situ hybridization was performed independently by the Dana Farber Cancer Institute In Situ Core Facility (Boston, Mass.). The Facility uses its own human tissue bank for this work. The plasmid was linearized with appropriate restriction enzymes and transcribed with T7 or T3 RNA polymerase (Promega, Madison, Wis.) and 35S-labeled UTP (New England Nuclear) to generate antisense and sense radiolabeled-RNA probes. Tissue sections were deparaffinized, fixed in 4% paraformaldehyde in PBS, and treated with proteinase K. After washing in 0.5×SSC, the sections were covered with hybridization solution (50% deionized formamide, 0.3M NaCl, 2 0 mM Tris (pH8.0), 5 mM EDTA, 1×Denhardt's solution, 10% Dextran Sulfate, and 10 mM Dithiothreitol) and prehybridized for two hours at 55° C. ³⁵S-labeled antisense and sense RNA probes (3×10⁵ cpm/slide) were added to the hybridization solution, and the incubation continued for 12-18 hours at 55° C. After hybridization, the sections were washed for 20 minutes in 2×SSC, 10 mM β-mercaptoethanol, and 1 mM EDTA, treated with RNAse A (10 μg/ml) for 30 minutes at room temperature, and washed at high stringency (0.1×SSC, 10 mM β-mercaptoethanol, and 1 mM EDTA) for two hours at 60° C. The sections were dehydrated, dipped in photographic emulsion NTB2 (Kodak), and stored at 4° C. After two weeks of exposure, the sections were developed and counterstained with hematoxylin and eosin.

Results.

Analysis of Predicted Sequence.

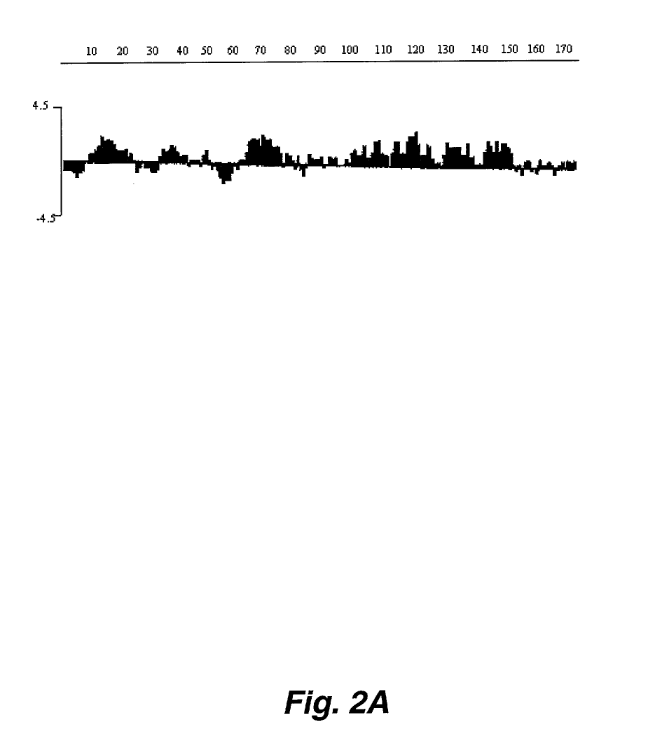

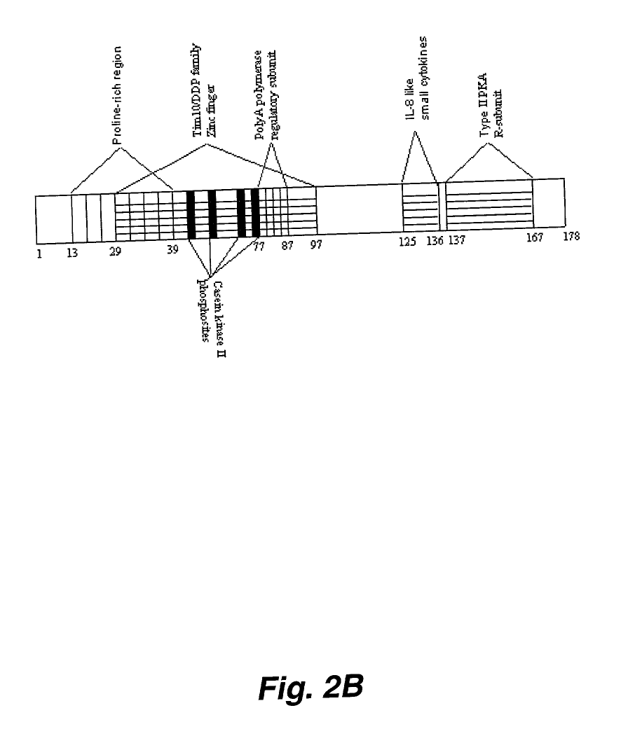

A BLASTN search in the Genbank database reveals that EG-1 (Genbank accession # AF358829) is on chromosome #4. It spans for exons (# 8-169, # 170-237, #238-349, # 350-1288) and three introns (5,087 base pairs; 1,619 bp; 1,901 bp). From the nucleotide sequence, the predicted peptide has 178 amino acids, and weighs 19.5 kD (FIG. 1A). There are 17 strongly basic amino acids, 21 strongly acidic amino acids, 60 hydrophobic amino acids, and 48 polar amino acids. The peptide has significant homology to a murine cDNA (94%, Genbank accession # NP_{—}080171, 12) and a Drosophila cDNA (31%, Genbank accession # AAF56470, 13) (FIG. 1B). It has no signal peptide nor transmembrane sequences (FIG. 2A). The isolectric point is 5.393, and the peptide has a −3.660 charge at pH 7.0. The melting temperature is 85° C.

A Profile Scan search reveals a long proline-rich region spanning from amino acid #13 to #39. There is one N-glycosylation site (aa # 66-69), four casein kinase II phosphorylation sites (aa # 43-46, 50-53, 68-71, 75-78), and two N-myristoylation sites (aa # 6-11, 76 81). A Pfam search looking for motif match show some alignment with the following: Tim 10/DDP (deafness dystonia protein) family zinc finger (aa # 29-97, E value 9.3), poly A polymerase regulatory subunit (aa # 77-87, E value 8.8), interleukin-8 like small cytokines (intecrine/chemokine) (aa # 125-136, E value 1.5), and regulatory subunit of type II PKA (cAMP-dependent protein kinase) R-subunit (aa # 137-167, E value 1.4) (FIG. 2B).

Northern Analysis of EG-1

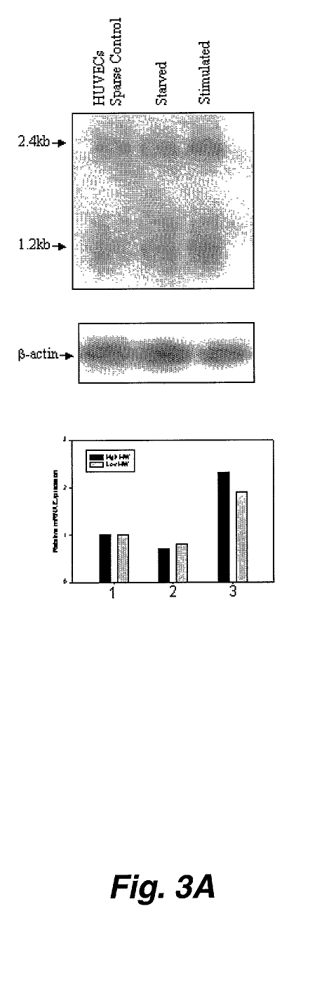

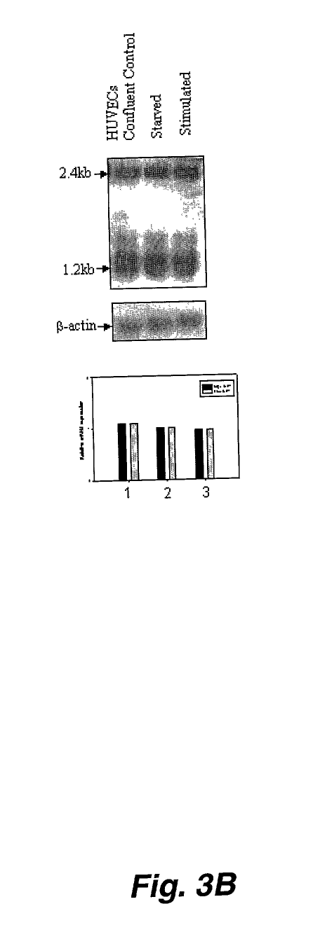

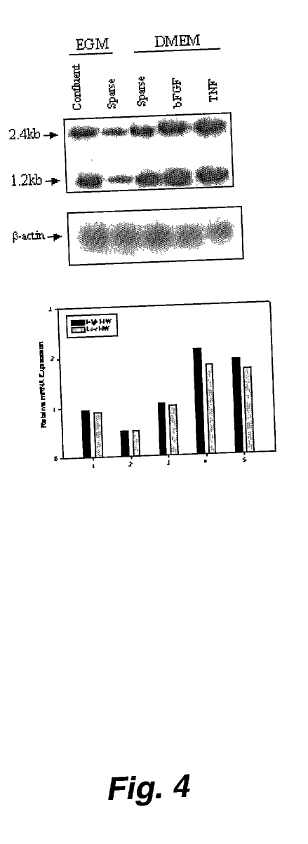

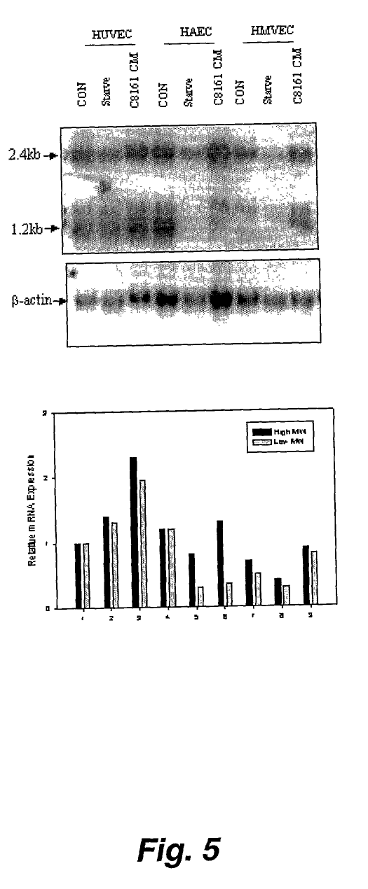

SSH revealed an RNA sequence (Genbank accession # AW735731), whose expression is increased in HUVECs treated with tumor conditioned media derived from either melanoma (C8161) or breast cancer (MDA-MB231). Subsequent cloning of the full length cDNA (Genbank accession # AF358829), and a BLASTN search for sequence homology performed in the Genbank database reveals that EG-1 has no significant homology to any gene with a known function. Northern analysis confirms that EG-1 expression is upregulated approximately two-fold in HUVECs exposed to tumor conditioned media (FIG. 3A). Two signals corresponding to a 2.4 kb and a second 1.2 kb are observed to both increase in intensity. The expression of EG-1 is unchanged when HUVECS are approaching confluency (90%-100% confluency) in culture (FIG. 3B). We then treated HUVECs to specific angiogenic factors. Stimulation with bFGF increases the expression of EG-1 by approximately two to three-fold, and TNFα by approximately two-fold (FIG. 4). When HUVECs are starved, the EG-1 transcript level decreases slightly (FIG. 5). The above observations are also seen in other types of endothelial cells including HAECs and HMVECs (FIG. 5). The increase in signal intensity due to exposure to tumor conditioned media is also observed in HAECs and HMVECs.

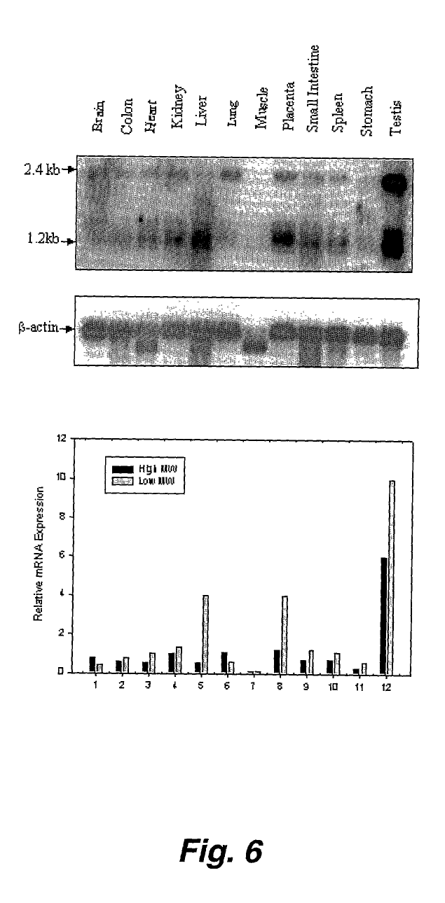

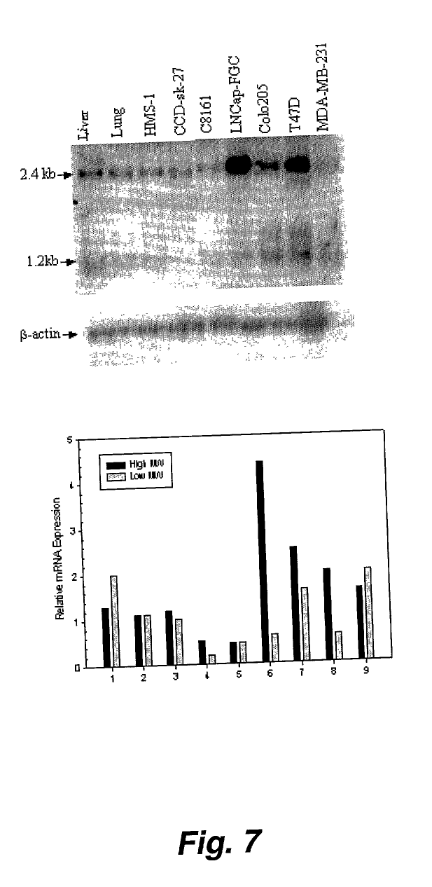

Further Northern studies of EG-1 show that it is highly expressed in liver, placenta, and testis (FIG. 6). The high expression is seen in both 2.4 kb and 1.2 kb forms in testis, but only in the lower m.w. 1.2 kb form in liver and placenta. When Northern analysis is performed with many different cells types, both m.w. forms can be detected (FIG. 7). These cell lines include benign types (fibroblast, myoepithelium, liver, and lung) as well as cancer cell lines derived from breast, colon, prostate, and melanoma. Interestingly, the higher m.w. 2.4 kb form is elevated in the breast cancer, colon cancer and prostate cancer cell lines.

In situ Hybridization of EG-1.

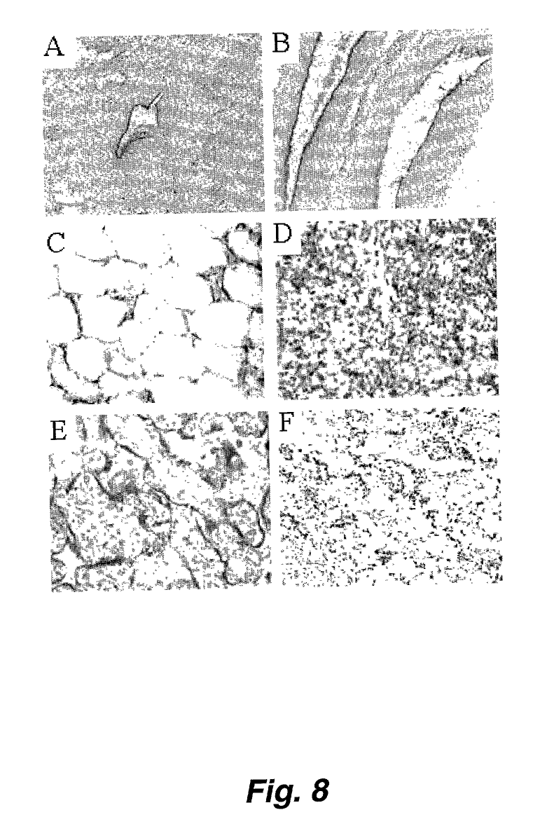

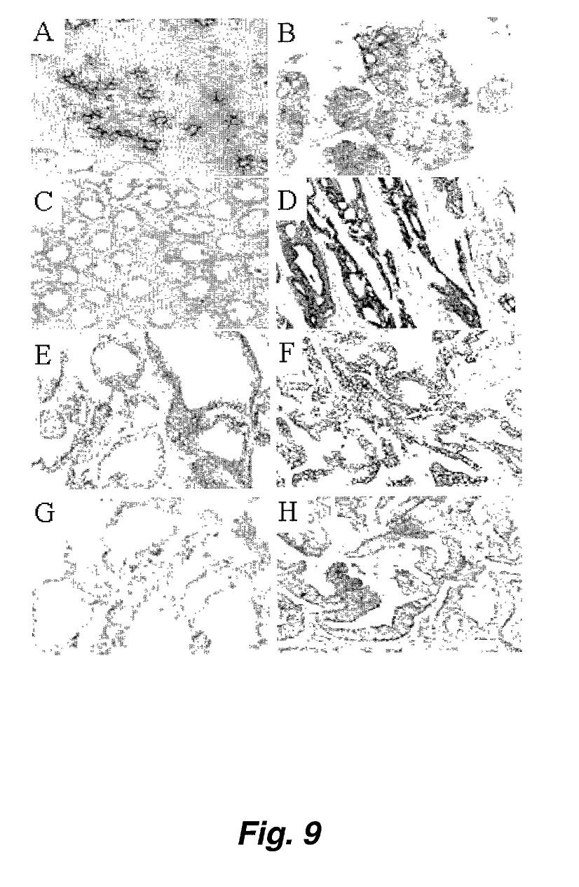

In situ hybridization of human tissues revealed staining of EG-1 in the endothelial cells of blood vessels. This is seen in arteries (FIG. 8A), veins (FIG. 8B), and capillaries (FIG. 8C). The signal is also detected in spleen endotheliocytes (FIG. 8D) and the placental Hoffbauer cells (FIG. 8E), which are presumed to be the precursor cells for endothelial cells, as well as in hemangioma blood vessels (FIG. 8F). We see the EG-1 signal in the epithelial cells of many organs, and this signal appears to be more intense with malignant transformation. Examples include breast cancer (FIGS. 9A-B), colon cancer (FIGS. 9C-D), prostate cancer (FIGS. 9E-F), and lung cancer (FIGS. 9G-H). No EG-1 signal is detected in lymphoid tissues (tonsils, thymus, lymph nodes, splenic lymphocytes), muscle (skeletal, smooth, cardiac, uterine), or fat (data not shown).

Discussion.

Endothelial-derived gene EG-1 seems to be a human gene, which has homology to both murine and Drosophila forms. From our Northern and in situ hybridization studies, it appears that EG-1 is expressed in endothelial cells. The expression of EG-1 seems to correlate with cellular proliferation or stimulation, as it is up-regulated by tumor conditioned media. Previously, we have seen that tumor conditioned media from C8161 and/or Mda-Mb-231 is rich with multiple angiogenic growth factors (Nguyen et al. (2000) Oncogene 19: 3449-3459). In this study, we further see that EG-1 expression is increased with exposure to two angiogenic factors bFGF and TNF-α.

Several researchers, including our laboratory, have investigated the difference between molecules of the proliferating tumor endothelium from those in the normal quiescent endothelium. One approach toward studying the tumor endothelium involves immunohistochemical analysis of known endothelial adhesion molecules using tumor specimens. These studies have shown that multiple surface molecules are significantly increased in the tumor vasculature. These molecules include E-selectin (Nguyen et al. (1997) Am. J. Path. 150: 1307-1310), the αVβ₃ integrin, VCAM-1 (vascular cellular adhesion molecule), ICAM-1 and -2 (intercellular adhesion molecule), CD 31, CD 34, CD 36, and CD 44 (Polverini (1996) Am. J. Path. 148: 1023-1029). Other investigators have used the antibody targeting approach. This approach has produced multiple candidate markers of the tumor vasculature. These include endoglin which is recognized by the TEC-11 antibody, endosialin which is recognized by the FB5 antibody, the antigen recognized by the EN7/44 antibody, the antigen recognized by the E-9 antibody (Thorpe and Burrows (1995) Breast Cancer Res. Treatm. 36: 237-251), a truncated form of tissue factor (Huang et al. (1997) Science 275: 547-550), and the fibronectin B-FN isoform (Neri et al. (1997) Nat. Biotechnol. 15: 1271-1275). Phage display peptide libraries have also been used successfully to characterize tumor blood vessels (Koivunen et al. (1999) Nat. Biotechnol. 17: 768-774). Differential RNA expression cloning has also been successfully pursued in endothelial cells treated with TPA (Lee et al. (1998) Science 279: 1552-1555) and in endothelial cells derived from colorectal cancer (St. Crox et al. (2000)Science 289: 1197-1202).

Recent reports of the effect of known angiogenic growth factors on the endothelium have advanced our understanding of the mechanisms of tumor angiogenesis at a molecular level. The best studied angiogenic growth factor is VEGF (vascular endothelial cell growth factor, Hanahan (1997) Science 277: 48-50). Other growth factors have been shown to be also important including bFGF, aFGF (acidic fibroblast growth factor), angiogenin, TGF-α and β (transforming growth factor alpha and beta), TNF-α, PD-ECGF (platelet derived endothelial growth factor), G-CSF (granulocyte colony stimulating factor), PlGF (placental growth factor), interleukin-8, HGF (hepatocyte growth factor), proliferin (Folkman (1995) N. Engl. J. Med. 333: 1757-1763), and angiopoietin (Suri et al. (1996) Cell 87: 1171-1180). Endogenous angiogenic inhibitors such as angiostatin, endostatin (O'Reilly et al. (1997) Cell 88: 277-285), thrombospondin, METH (Vazquez et al. (1999) J. Biol. Chem. 274: 23349-23357) may also play an important role in this process. Proteasess and cytokines secreted by tumor cells are also very important.

In our laboratory, we used SSH to further investigate the molecular mechanisms of tumor angiogenesis by identifying genes that become activated as well as those that become down-regulated when quiescent endothelial cells are exposed to a tumor environment. Although this project utilizes cells in tissue culture, we think that this in vitro model does provide an adequate simulation of the tumor environment. With this model, we have recently identified human endomucin (Liu et al. (2001) Biochem. Biophys. Res. Commun. 288: 129-136). Other investigators have used similar methods of differential display to study non-cancer related in vitro models of angiogenesis and have found increased expression of important angiogenesis-related genes such as endothelial differentiation gene (Lee et al. (1998) Science 279: 1552-1555) and COX-1 (cyclooxygenase, Narko et al. (1997) J. Biol. Chem. 272: 21455-21460).

The function of EG-1 was previously unknown. The molecule shows significant homology to a murine and a Drosophila form, whose functions are also unknown. Based on our own sequence analysis, EG-1 might be involved in signal transduction. The presence of four casein kinase II phosphorylation sites indicates that EG-1 might have the capacity to be a signaling molecule. EG-1 also shows some motif alignment with the Tim 10/DDP family zinc finger, the poly A polymerase regulatory subunit, the small IL-8-like cytokines, and the regulatory subunit of type II PKA R-subunit.

Without being bound by a particular theory, we believe EG-1 has a role in one or more steps of angiogenesis such as endothelial proliferation, migration or differentiation into tube-like structures. Consequently, EG-1 can potentially be targeted in the treatment and diagnosis of human disease. Utility is seen in many angiogenesis-related diseases including heart disease and stroke, as well as in cancer.

### Example 2

### Effects of EG-1 Inhibition

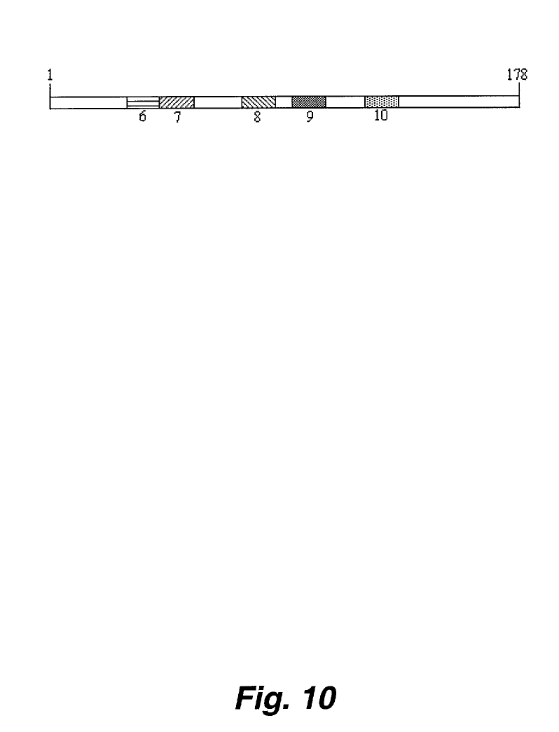

In this example we examined the effects of EG-1 inhibition using anti-EG-1 antibodies or EG-1 peptides. The EG-1 peptides and antibodies used in this study are identified in Table 1 and their position on EG-1 is illustrated in FIG. 10.

TABLE 1
EG-1 peptides and location on EG-1.

| | | | Amino | |
|---|---|---|---|---|
| | | Antibody | acid | SEQ |
| Peptide | | to | position in | ID |
| Number | Sequence | sequence | EG-1 | NO |
| 6 | APPGLPGQASLLQAAPG | Yes | 19-35 | 11 |
| 7 | PGAPRPSSSTLVDELESSFE | Yes | 34-53 | 12 |
| 8 | IRTGVDQCIQKFLDIAR | Yes | 73-89 | 13 |
| 9 | CFFLQKRLQLSVQKPEQV | Yes | 93-110 | 14 |
| 10 | ELQRKDALVQKHLTKLR | Yes | 121-137 | 15 |

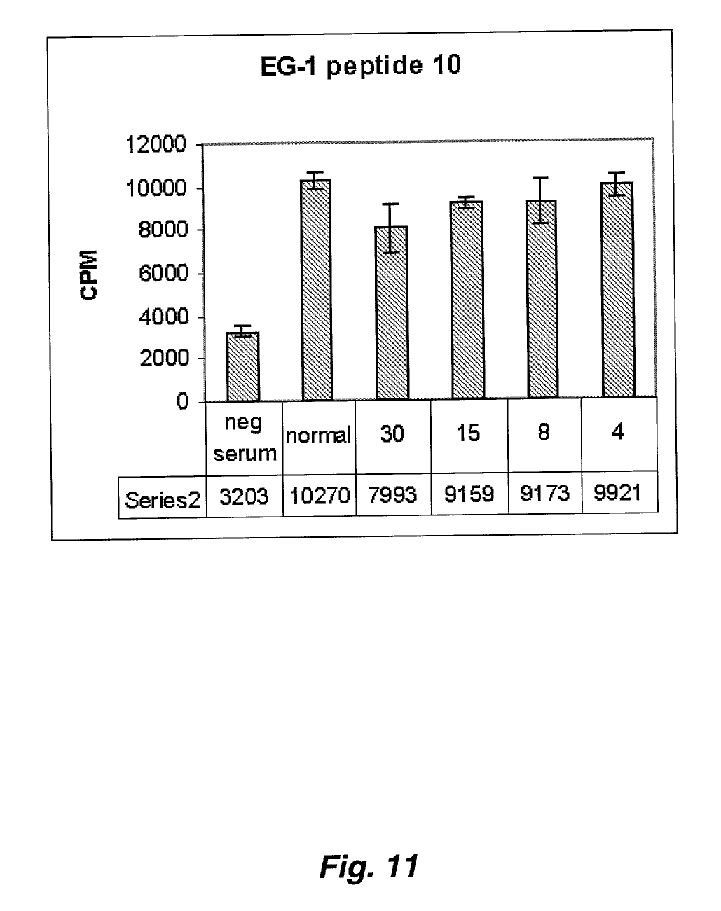

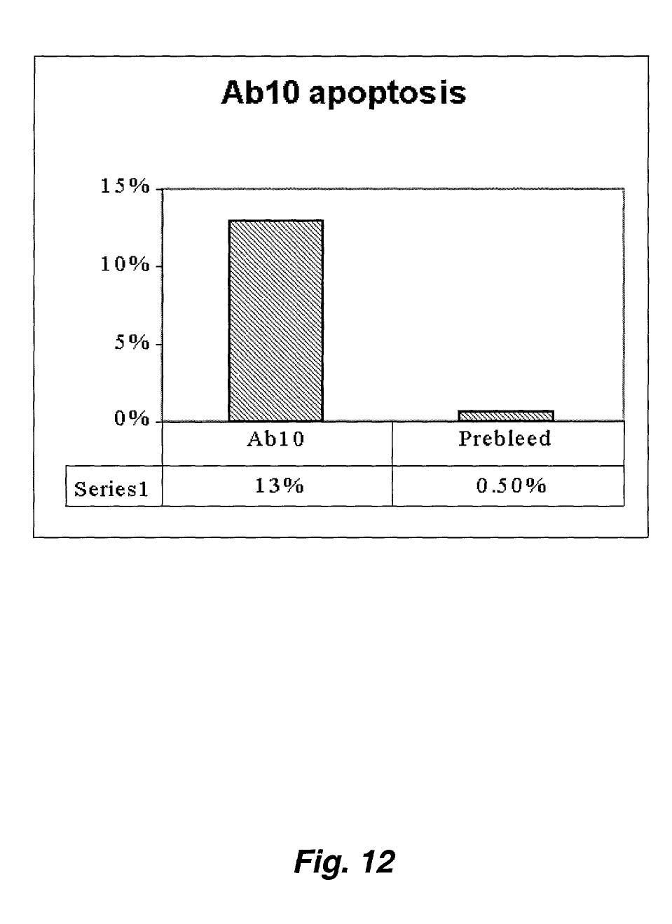

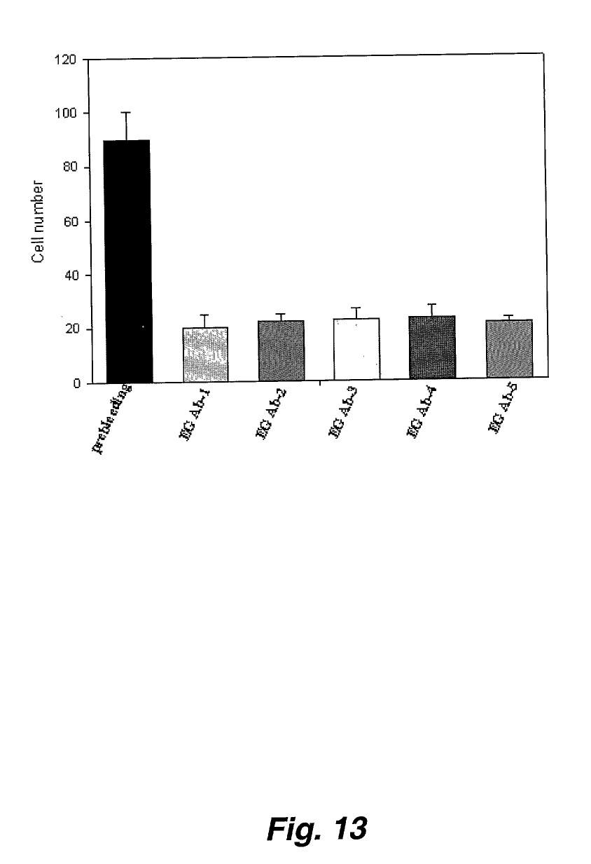

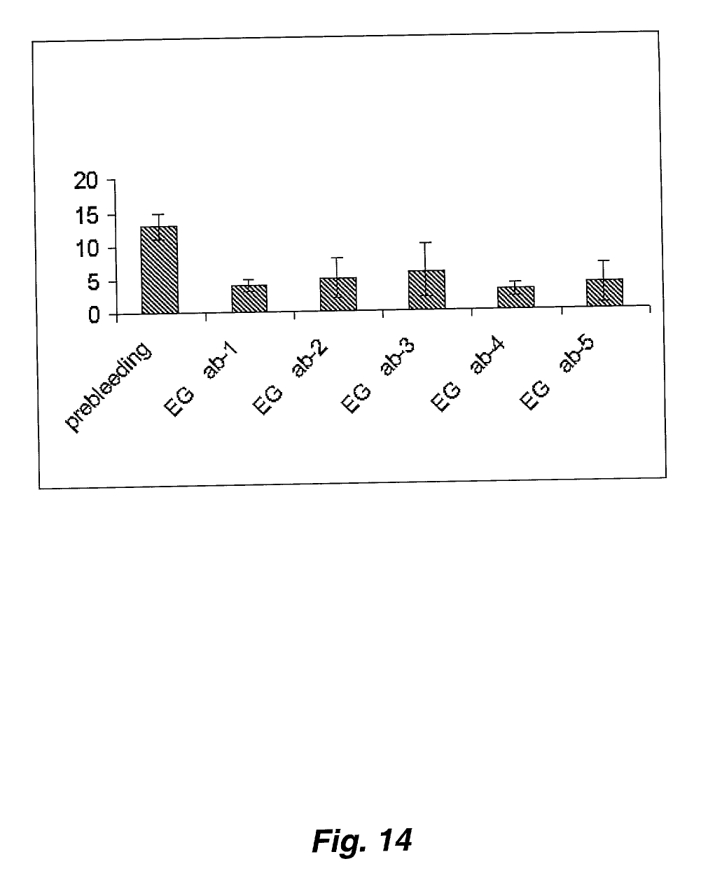

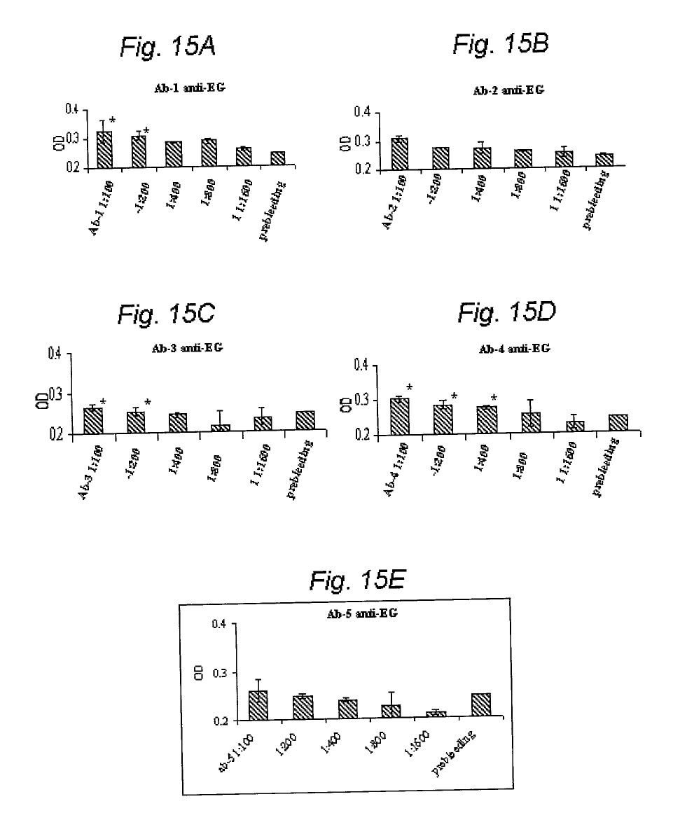

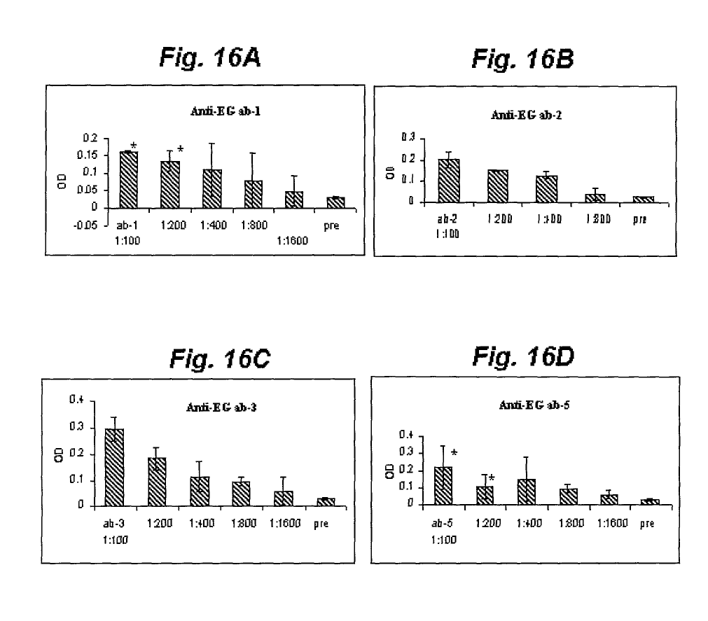

The data presented herein demonstrate that EG-1 has a function in angiogenesis. Interference with antibodies or peptide fragments cause an inhibition in endothelial cell proliferation (FIG. 11) as assayed according the methods of Nguyen et al. (1996) Biochem. Biophys. Res. Commun. 228: 716-23; Nguyen et al. (2000). Oncogene 19: 3449-3459; and Sartippour et al. (2001) Oncol. Rep. 8: 1355-1357. Antibodies or peptide fragments also cause an increase in apoptosis (FIG. 12) as assayed using the apoptag kit made by Intergen. Antibodies or peptide fragments additionally inhibit endothelial migration and tube formation (FIGS. 13 and 14) as assayed, e.g. according to the methods described by Nguyen et al. (1992) J. Biol. Chem. 267:26157-16165. All of thes processes are important steps in the process of angiogenesis. Also, EG 1 is involved in the adhesion between endothelial cells and cancer cells (FIGS. 15A-15D). This is important in cancer metastasis. Interference with antibodies inhibits adhesion in breast and colon cancer cells (FIGS. 16A-16A), e.g. as assayed according to the methods of Tomlinson et al. (2000). Int. J. Oncol. 16:347-353,

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated nucleic acid comprising a nucleic acid selected from the group consisting of:
(i) a nucleic acid that specifically hybridizes to a human EG-1 cDNA (coding region of SEQ ID NO:1) or a fragment thereof under stringent conditions and that is of sufficient length that said nucleic acid can uniquely indicate the presence or absence of a human EG-1 total genomic DNA pool, a total cDNA pool or a total mRNA pool sample from an endothelial cell;
ii) a nucleic acid that encodes a human EG-1 polypeptide (SEQ ID NO:2);
(iii) a nucleic acid that has the same sequence as a nucleic acid amplified from an endothelial cell mRNA template using PCR primers Primer-1 (SEQ ID NO:7) and Primer-2 (SEQ ID NO:8), or Primer-3 (SEQ ID NO:9) and Primer-4 (SEQ ID NO:10);
(iv) a DNA encoding an mRNA that, when reverse transcribed, produces a human EG-1 cDNA (coding region of SEQ ID NO:1);
v) a nucleic having 90 percent or greater sequence identity with a human EG-1 nucleic acid (coding region of SEQ ID NO:1) and encoding a polypeptide, expression of which is upregulated in an epithelial tumor cell;
(vi) a pair of primers that, when used in a nucleic acid amplification reaction with an endotheial cell mRNA template specifically amplifies a nucleic acid encoding a human EG-1 polypeptide (SEQ ID NO:2).

2. The nucleic acid of claim 1, wherein said nucleic acid encodes a polypeptide having the sequence of SEQ ID NO:2.

3. The nucleic acid of claim 1, wherein said nucleic acid comprises the sequence of the coding region of SEQ ID NO:1.

4. The nucleic acid of claim 1, wherein said nucleic is present in a vector.

5. The nucleic acid of claim 1, wherein said nucleic acid is at least 15 nucleotides in length.

6. The nucleic acid of claim 5, wherein said nucleic acid is labeled with a detectable label.

7. The nucleic acid of claim 6, wherein said detectable label is selected from the group consisting of a magnetic label, a radioactive label, a colorimetric label, and a fluorescent label.

8. A polypeptide encoded by a nucleic acid of claim 1, wherein expression of said polypeptide is upregulated in an endothelial cell.

9. A polypeptide encoded by a nucleic acid of claim 1, wherein expression of said polypeptide is upregulated in an epithelial cell cancer.

10. A polypeptide comprising the amino acid sequence of SEQ ID NO:2 or conservative substitutions thereof.

11. A cell transfected with a nucleic acid of claim 1, wherein said nucleic acid encodes an EG-1 polypeptide.

12. An antibody that specifically binds to a peptide comprising the amino acid sequence of SEQ ID NO:2.

13. The antibody of claim 12, wherein said antibody is a polyclonal antibody.

14. The antibody of claim 12, wherein said antibody is a single-chain antibody.

15. A method of screening for a test agent that modulates tissue angiogenesis or tumorigenesis said method comprising:
contacting a cell comprising an EG-1 gene with a test agent; and
detecting a change in the expression or activity of an EG-1 gene product as compared to the expression or activity of a EG-1 gene product in a control cell, where a difference in the expression or activity of EG-1 in the contacted cell and the control cell indicates that said agent alters tissue angiogenesis.

16. The method of claim 15, wherein said control cell is the same type of cell exposed to a lower concentration of test agent.

17. The method of claim 16, wherein said lower concentration is the absence of said test agent.

18. The method of claim 15, wherein said cell is an endothelial cell.

19. The method of claim 15, wherein said cell is an epithelial cell.

20. The method of claim 15, wherein the expression of EG-1 gene product is detected by detecting EG-1 mRNA in said sample.

21. The method of claim 20, wherein the level of EG-1 mRNA is measured by hybridizing said mRNA to a probe that specifically hybridizes to an EG-1 nucleic acid.

22. The method of claim 21, wherein said hybridizing is according to a method selected from the group consisting of a Northern blot, a Southern blot using DNA derived from the EG-1 RNA, an array hybridization, an affinity chromatography, and an in situ hybridization.

23. The method of claim 21, wherein said probe is a member of a plurality of probes that forms an array of probes.

24. The method of claim 20, wherein the level of EG-1 mRNA is measured using a nucleic acid amplification reaction.

25. The method of claim 15, wherein the expression of EG-1 gene product is detected by detecting the level of an EG-1 protein in said biological sample.

26. The method of claim 25, wherein said detecting is via a method selected from the group consisting of capillary electrophoresis, a Western blot, mass spectroscopy, ELISA, immunochromatography, and immunohistochemistry.

27. The method of claim 15, wherein said cell is cultured ex vivo.

28. The method of claim 15, wherein said test agent is contacted to an animal comprising a cell containing the EG-1 nucleic acid or the EG-1 protein.

29. A method of prescreening for an agent that modulates tissue angiogenesis or tumorigenesiss, said method comprising:
i) contacting an EG-1 nucleic acid or an EG-1 protein with a test agent; and
ii) detecting specific binding of said test agent to said EG-1 protein or nucleic acid wherein specific binding indicates that said agent is a candidate modulator of tissue angiogenesis or tumorigenesis.

30. The method of claim 29, further comprising recording test agents that specifically bind to said EG-1 nucleic acid or protein in a database of candidate agents that modulate tissue angiogenesis or tumorigenesis.

31. The method of claim 29, wherein said test agent is not an antibody.

32. The method of claim 29, wherein said test agent is not a protein.

33. The method of claim 29, wherein said test agent is not a nucleic acid.

34. The method of claim 29, wherein said test agent is a small organic molecule.

35. The method of claim 29, wherein said detecting comprises detecting specific binding of said test agent to said EG-1 nucleic acid.

36. The method of claim 35, wherein said binding is detected using a method selected from the group consisting of a Northern blot, a Southern blot using DNA derived from a EG-1 RNA, an array hybridization, an affinity chromatography, and an in situ hybridization.

37. The method of claim 29, wherein said detecting comprises detecting specific binding of said test agent to said EG-1 protein.

38. The method of claim 37, wherein said detecting is via a method selected from the group consisting of capillary electrophoresis, a Western blot, mass spectroscopy, ELISA, immunochromatography, and immunohistochemistry.

39. The method of claim 29, wherein said test agent is contacted directly to the EG-1 nucleic acid or to the EG-1 protein.

40. The method of claim 29, wherein said test agent is contacted to a cell containing the EG-1 nucleic acid or the EG-1 protein.

41. The method of claim 40, wherein said cell is cultured ex vivo.

42. The method of claim 29, wherein said test agent is contacted to an animal comprising a cell containing the EG-1 nucleic acid or the EG-1 protein.

43. A transgenic animal comprising a recombinantly modified EG-1 gene such that said recombinantly modified gene does not transcribe a functional EG-1 protein.

44. The transgenic animal of claim 43, wherein said animal is homozygous for said recombinantly modified EG-1 gene.

45. The transgenic animal of claim 43, wherein said animal is a murine.

46. The transgenic animal of claim 43, wherein said animal is a mouse.

47. The transgenic animal of claim 43, wherein said animal is chimeric for cells comprising said recombinantly modified EG-1 gene.

48. A method of identifying a predilection to developing one or more symptoms of a disease characterized by abnormal angiogenesis, said method comprising:
obtaining a biological sample from said organism; and
detecting overexpression of an EG-1 gene product.

49. A method of inhibiting angiogenesis, said method comprising inhibiting the expression or activity of an EG-1 gene product/

50. The method of claim 49, wherein the inhibiting is by a method selected from the group consisting of contacting an EG-1 nucleic acid with a ribozyme that specifically cleaves the EG-1 nucleic acid, contacting an EG-1 nucleic acid with a catalytic DNA that specifically cleaves the EG-1 nucleic acid, transfecting a cell comprising an EG-1 gene with a nucleic acid that inactivates the EG-1 gene by homologous recombination with the EG-1 gene, transfecting a cell comprising a with a nucleic acid encoding an intrabody that specifically binds an EG-1 polypeptide, transfecting a cell comprising an EG-1 gene with an EG-1 antisense molecule, contacting a cell with an EG-1 polypeptide or fragment thereof, and contacting an EG-1 polypeptide with an antibody that specifically binds the EG-1 polypeptide.

51. The method of clam 50, wherein said inhibiting comprises contacting an EG-1 polypeptide with an antibody that specifically binds the EG-1 polypeptide.

52. The method of claim 51, wherein said antibody specifically binds an EG-1 fragment selected from the EG-1 fragments listed in Table 1.

53. The method of claim 51, wherein said antibody is a polyclonal antibody.

54. The method of claim 51, wherein said antibody is a single-chain antibody.


SEQUENCE LISTING (15 sequences)

SEQ ID NO: 1 - DNA, 1281 bp, Homo sapiens
  CDS (6)..(539)
caaacatggcggctccactagggggtatgttttctgggcagccacccggtccccctcaggccccgccgggccttccgggccaagcttcgcttcttcaggcagctccaggcgctcctagaccttccagcagtactttggtggacgagttggagtcatctttcgaggcttgctttgcatctctggtgagtcaggactatgtcaatggcaccgatcaggaagaaattcgaaccggtgttgatcagtgtatccagaagtttctggatattgcaagacagacagaatgttttttcttacaaaaaagattgcagttatctgtccagaaaccagagcaagttatcaaagaggatgtgtcagaactaaggaatgaattacagcggaaagatgcactagtccagaagcacttgacaaagctgaggcattggcagcaggtgctggaggacatcaacgtgcagcacaaaaagcccgccgacatccctcagggctccttggcctacctggagcaggcatctgccaacatccctgcacctctgaagccaacgtgagcaaagggcagaggcagttggcctatgagtgggctgatgcgtgaggttggccacacattccttcctgtggacttgacattttggaagaactctttgccagataatgagttcattttagttttatgctcccattgaaaaattttccactatttttataagctgttaatttcttgagtactttataacatgtctgtagcttggataaaccaagtaagtattttttttttgtctttagcaaagtttagactgtgaatatgatgacacagattcttttttatggtggctttgcttgttttaaatttttgcatgacttttcatctttttatgtgtgtttcctgtagtttgatccgaaggaaaagagtatagtagcctgagaatcaggagatgggagttttagtcgtaggccttatgataattaccccgcggtggtgtgtagaaaagtatgtaaatttgctctgttttaagactttgaactacctcaagaagaggaatctaatacaatatttgtaatgtttccagagctctcagaatgaggatttttttgtaaataggtcagaagacgatggaactgtcctgggttagtatagtaatcttacagtaggatccttaggttgatgctgacttctgtttggggtatgtttatattttatgtggtgtttactttttttttttgacataaaaggatatagtgggagcagtgatacgctaacattcattacattctgcagtaatgaatctg

SEQ ID NO: 2 - protein, 178 aa, Homo sapiens
MAAPLGGMFSGQPPGPPQAPPGLPGQASLLQAAPGAPRPSSSTLVDELESSFEACFASLVSQDYVNGTDQEEIRTGVDQCIQKFLDIARQTECFFLQKRLQLSVQKPEQVIKEDVSELRNELQRKDALVQKHLTKLRHWQQVLEDINVQHKKPADIPQGSLAYLEQASANIPAPLKPT

SEQ ID NO: 3 - protein, 178 aa, Mus musculus
MAASLGGMFTGQPPGPPPPPPGLPGQASLLQAAPGAPRPSNSTLVDELESSFEACFASLVSQDYVNGTDQEEIRTGVDQCIQKFLDIARQTECFFLQKRLQLSVQKPDQVIKEDVSELRSELQRKDALVQKHLTKLRHWQQVLEDINVQHKKPADMPQGSLAFLEQASANIPAPLKQT

SEQ ID NO: 4 - protein, 189 aa, Drosophila melanogaster
MASNESGGGNLMDEFEEAFQSCLLTLTKQEPNSGTNKEEIDLEVQKTTNRFIDVARQMEAFFLQKRFLVSTLKPYMLIKDENQDLSIEIQRKEALLQKHYNRLEEWKACLSDIQQGVHSRPTPPIGSGMLQGPGGGMPPMGGTPPRPGMMPGMPPGAMQPGGPMQPSPHMLQAQQMQQLRMISRQMPPK

SEQ ID NO: 5 - DNA, 1363 bp, Homo sapiens
  CDS (13)..(546)
ggcacagcaaacatggcggctccactagggggtatgttttctgggcagccacccggtccccctcaggccccgccgggccttccgggccaagcttcgcttcttcaggcagctccaggcgctcctagaccttccagcagtactttggtggacgagttggagtcatctttcgaggcttgctttgcatctctggtgagtcaggactatgtcaatggcaccgatcaggaagaaattcgaaccggtgttgatcagtgtatccagaagtttctggatattgcaagacagacagaatgttttttcttacaaaaaagattgcagttatctgtccagaaaccagagcaagttatcaaagaggatgtgtcagaactaaggaatgaattacagcggaaagatgcactagtccagaagcacttgacaaagctgaggcattggcagcaggtgctggaggacatcaacgtgcagcacaaaaagcccgccgacatccctcagggctccttggcctacctggagcaggcatctgccaacatccctgcacctctgaagccaacgtgagcaaagggcagaggcagttggcctatgagtgggctgatgcgtgaggttggccacacattccttcctgtggacttgacattttggaagaactctttgccagataatgagttcattttagttttatgctcccattgaaaaattttccactatttttataagctgttaatttcttgagtactttataacatgtctgtagcttggataaaccaagtaagtattttttttttgtctttagcaaagtttagactgtgaatatgatgacacagattcttttttatggtggctttgcttgttttaaatttttgcatgacttttcatctttttatgtgtgtttcctgtagtttgatccgaaggaaaagagtatagtagcctgagaatcaggagatgggagttttagtcgtaggccttatgataattaccccgcggtggtgtgtagaaaagtatgtaaatttgctctgttttaagactttgaactacctcaagaagaggaatctaatacaatatttgtaatgtttccagagctctcagaatgaggatttttttgtaaataggtcagaagacgatggaactgtcctgggttagtatagtaatcttacagtaggatccttaggttgatgctgacttctgtttggggtatgtttatattttatgtggtgtttactttttttttttgacataaaaggatatagtgggagcagtgatacgctaacattcattacattctgcagtaatgaatctgaaaaaaaaaaaaaaaaaaaaaaaaaaaaaaaaaaaaaaaaaaaaaaaaaaaaaaaaaaaaaaaaaaaaaaaaaaa

SEQ ID NO: 6 - protein, 178 aa, Homo sapiens; identical to SEQ ID NO: 2

SEQ ID NO: 7 - DNA, 18 bp, Artificial Sequence
  PCR primer
tcacgttggcttcagagg

SEQ ID NO: 8 - DNA, 18 bp, Artificial Sequence
  PCR primer
atggcggctccactaggg

SEQ ID NO: 9 - DNA, 18 bp, Artificial Sequence
  PCR primer
tcacgttggcttcagagg

SEQ ID NO: 10 - DNA, 22 bp, Artificial Sequence
  PCR primer
caccatggcggctccactaggg

SEQ ID NO: 11 - protein, 17 aa, Homo sapiens
APPGLPGQASLLQAAPG

SEQ ID NO: 12 - protein, 20 aa, Homo sapiens
PGAPRPSSSTLVDELESSFE

SEQ ID NO: 13 - protein, 17 aa, Homo sapiens
IRTGVDQCIQKFLDIAR

SEQ ID NO: 14 - protein, 18 aa, Homo sapiens
CFFLQKRLQLSVQKPEQV

SEQ ID NO: 15 - protein, 17 aa, Homo sapiens
ELQRKDALVQKHLTKLR